(19) United States
(12) Patent Application Publication
(10) Pub. No.: US 2026/0283859 A1
(43) Pub. Date: Sep. 24, 2026

(54) REDUCED-LEAKAGE TAMPON

(71) Applicant: TAMPRO INC., San Francisco, CA (US)

(72) Inventors: Greta Catherine Meyer, San Francisco, CA (US); Amanda Paige Calabrese, San Francisco, CA (US); Elijah John Sampson Zenger, New York, NY (US); David L. Kimball, Flemington, NJ (US)

(21) Appl. No.: 19/478,462

(22) PCT Filed: Apr. 26, 2024
(86) PCT No.: PCT/US2024/026537

§ 371 Date: Oct. 24, 2025

Related U.S. Application Data
(60) Provisional application No. 63/499,143, filed on Apr. 28, 2023.

(51) Int. Cl. A61F 13/20

(52) U.S. Cl. CPC A61F 13/2091; A61F 13/2034; A61F 13/2088

(57) ABSTRACT

Tampons and methods of manufacturing the tampons. The tampons can include an elongate body and a removal string extending from a removal end of the elongate body. The tampons can include a flow path that directs menses across the elongate body. The tampons can include a plurality of longitudinal grooves crossing the flow path.

### BACKGROUND

### Field

The present disclosure relates to tampons for feminine hygiene and other uses.

### Description of the Related Art

Tampons are most commonly used to retain menstrual fluid and tissue. The fluid and tissue is absorbed and held by the tampon for a certain amount of time. After this period, the tampon is removed and a new tampon is used if the user desires.

### SUMMARY

Tampons are often made of compressed absorbent fibers with a cover layer. Existing tampons include longitudinal grooving or channeling on the outside of tampons to direct a flow of menses (see FIG. 1). Within the space, however, there is still a significant issue with tampons leaking before reaching significant levels of saturation, discomfort, pain, pressure, and/or not performing as well as necessary. This can be particularly stressful in a public setting, for example at the workplace or on the athletic field.

Tampons with the absorbent materials and flow paths described herein produce, in several embodiments, a more efficiently and evenly absorbing tampon. In several embodiments, the absorbent material absorbs the less viscous menstrual material, while the flow paths redirect the more viscous menstrual material as it travels down the elongate body of the tampon in order to give it more time to absorb. Although several tampons described herein are for menstruation, the tampons described herein may also be used for other applications such as nose bleeds.

In several embodiments, the tampons described herein have one or more of the following advantages:
slow the total travel time for material (such as menstrual material) across the tampon giving the tampon more time to absorb the material;
reduce the likelihood of possible leakage before full saturation of the tampon;
require less frequent replacement of tampons and lower the total cost of goods required per period cycle;
conform to the rugae along the target regions (such as vaginal walls) to prevent displacement during active movement;
sustainable materials, and/or
absorb clots and more viscous material.

The tampon can include an elongate body comprising an outer surface, an insertion end, and a removal end. The elongate body can include first region at an insertion end and a second region at a removal end. The elongate body can include a transition region therebetween. A diameter of the first region can be different than a diameter of the second region, for example the diameter of the first region can be greater than the diameter of the second region. The tampon can include a plurality of longitudinal recesses in the outer surface of the elongate body. Each of the plurality of longitudinal recesses can extend from the first region to the second region. The tampon can include a plurality of wells configured to allow fluid to flow toward a core of the elongate body. Each of the plurality of wells can extend from one of the plurality of longitudinal recesses to an adjacent one of the plurality of longitudinal recesses. The plurality of wells can be interconnected through the plurality of longitudinal recesses to form a spiral recess. The tampon can have at least 24 wells, at least 28 wells, or at least 32 wells.

Each of the plurality of wells has a depth measured from the outer surface toward the core of the elongate body and a height measured in a longitudinal direction at the outer surface of the elongate body. The height of each of the plurality of wells can be tapered from the outer surface of the elongate body towards the core. The depth of each of the plurality of wells can be at least 10% and/or less than or equal to 20% of a diameter of the elongate body. The depth of each of the plurality of wells can be at least about 0.5 mm and/or less than or equal to about 2.0 mm. The depth can be between about 0.5 mm to about 1.0 mm, between 0.75 to about 1.25 mm, between about 1.0 mm to about 1.5 mm, between about 1.25 mm to about 1.75 mm, or between about 1.5 mm to about 2.0 mm. The depth can be equal to the height, less than the height, or greater than the height.

The tampon can include an elongate body having a first region at an insertion end, a second region at a removal end, and a transition region therebetween. The first region can be offset compared to the second region. For example, the first region can include a different diameter compared to a diameter of the second region, e.g., at least 5% greater, or at least 10% greater, than the diameter of the second region. In other implementations, the diameter of the first region is no larger than a diameter of the second region. The first region can be dome-shaped, cone-shaped, or any other shape. A length of the first region can be less than 20%, or less than 15%, of a length of the elongate body. The transition region can include an undercut or step-like feature.

In some embodiments, tampons that vary in diameter and/or have an offset, flare or step are particularly beneficial because they can, for example, increase or maintain absorption while still conforming securely to the anatomy without causing (or at least reducing) pressure, pain or discomfort during insertion, wear and/or removal. In several embodiments the diameter is constant for 70-95% of the length of tampon and changes for the remaining 5-30%. For example, a tampon having a length of about 4 cm may be constant in diameter for 3-4 cm and include a larger portion of 0.5-1 cm. The larger diameter may be at the bottom of the tampon (near the optional string) or at (or near) the top. In other embodiments, the diameter varies along over at least 50%, 75% or 95% of the tampon or along its entire length (such as a cone shape, with the larger portion being at or near the bottom or top). The portion with the larger diameter may be 5-30% (e.g., 5-10%, 10-20%, 15-30% and overlapping ranges therein) larger the portion of the tampon with the smallest diameter. The larger portion may be constructed of the same or different material than the other portions. The larger portion may be have the same or different absorbency than the other portions.

Any of the tampons described herein can include a spiral flow path (e.g., recess or protrusion) in an outer surface of the elongate body. The spiral flow path can turn about a longitudinal axis of the elongate body and extend at least one complete turn around a circumference of the elongate body. The spiral flow path can form a generally continuous flow path. The spiral flow path could be entirely continuous or intermittent but sufficiently continuous to allow continuous flow along the spiral flow path. A depth of the spiral flow path can be substantially constant along a length of the spiral flow path. For example, the depth can vary by no more than 10% or 5% along a length of the spiral flow path. In other configurations, the depth of the spiral flow path may vary. For example, the spiral flow path may be formed by a series of discrete segments (e.g., indentations). A first end of the spiral flow path can be positioned in the first region, and a second end of the spiral recess can be positioned at or near the removal end. The spiral flow path can cross the transition region. For example, the transition region can be positioned between a first location at an end of the spiral recess and a second location one complete turn from the first location. Each turn of the spiral flow path can be angled closer to horizontal than the longitudinal axis of the elongate body. The pitch of the spiral flow path can be greater than a length of the first region.

Any of the tampons described herein can include a plurality of longitudinal flow paths (e.g., recesses or ribs) in the outer surface of the elongate body. Where a spiral flow path is present, each of the plurality of longitudinal recesses can cross successive turns of the spiral recess. A first end of each of the plurality of longitudinal recesses can be in the first region. A second end of each of the plurality of longitudinal recesses can be at or near the removal end. Each longitudinal groove may be defined by a first wall extending in a radial direction and a second wall extending in a radial direction. The first wall and the second wall can be non-arcuate walls. The first wall and the second wall can generally meet at an apex thereby forming a generally triangular cutout when the tampon is viewed in cross-section. The plurality of longitudinal recesses cross the spiral recess to form at least several intersections, for example at least 24 intersections, at least 28 intersections, at least 32 intersections, or at least 36 intersections. 9. The tampon of claim 5, wherein a combination of the plurality of longitudinal recesses and the spiral recess form at least 48 cm of recessed flow paths in the tampon. A combination of the plurality of longitudinal recesses and the spiral recess can extend across at least 15%, at least 20%, or at least 30% of a surface area of the elongate body.

The tampons herein can include a plurality of wells configured to allow fluid to flow toward a core of the elongate body. Each of the plurality of wells extending from one longitudinal recess to an adjacent longitudinal recess. Each of the plurality of wells can be longitudinal offset from a circumferentially adjacent one of the plurality of wells. The elongate body can include a plurality of lobes formed by a plurality of longitudinal recesses. Each lobe can include a plurality of longitudinally spaced apart wells. The plurality of wells can include at least 24 wells and/or less than or equal to 48 wells, for example at least 28 wells, at least 32 wells, or at least 36 wells. A depth of each well, measured from an outer surface toward the core, can be at least 10% of a diameter of the elongate body. For example, the depth can be at least about 1.0 mm and/or less than or equal to about 3.0 mm, for example at least 1.5 mm, or at least 2.0 mm.

It can be difficult to manufacture a large volume of tampons with multiple flow paths. The manufacturing methods described herein include a single press capable of forming multiple flow paths, which may have different shapes, locations, and/or arrangements. In some embodiments, the multiple flow paths are simultaneously formed in the body of the tampon. With different mold segments forming multiple flow paths, there can be issues with transferring the tampon out of the press. The tampon presses and methods of manufacturing described herein improve the speed and efficiency at which the tampons are produced.

Tampons described herein can be formed using a press. The press can include a first set of jaws configured to form a flow path in an elongate body and/or a second set of jaws configured to form a flow path in the elongate body. In some arrangements, the first set of jaws and the second set of jaws form different types of flow paths (e.g., different shapes, locations, depths, and/or otherwise). For example, the first set of jaws can form a spiral recess and the second set of jaws can form a plurality of longitudinal grooves. Each set of jaws may form only one type of flow path. The flow path(s) formed by each jaw extend in the same direction. In other words, there may be no jaws forming both the first flow path and the second flow path. Each jaw may form a linear path or indentation (also referred to herein as wells) in the tampon, not a dimple or non-linear indentation. Each path or indentation may be formed by straight lines, which may be extending in generally the same direction. There are no indentations or paths extending in opposite directions. The first set of jaws may form a plurality of indentations or wells extending in the same direction. The indentations or wells formed by each jaw may be parallel to each other. There are no indentations extending in different directions. In other arrangements, the first set of jaws and the second set of jaws may form portions of the same flow path. The first set of jaws and the second set of jaws can include the same number of jaws, for example eight jaws.

The first set of jaws and the second set of jaws may be arranged in an alternating arrangement. The first set of jaws and the second set of jaws can be circumferentially arranged. Each of the first set of jaws can include a plurality of projections extending in a radial direction. The arrangement of the plurality of projections can differ between one or more of the first set of jaws. In some embodiments, each jaw of the first set of jaws is unique. The press can include the second set of jaws. Each jaw of the second set of jaws can include a straight edge configured to form the plurality of longitudinal recesses. Each jaw of the second set of jaws can include a first surface and a second surface positioned at an angle relative to the first surface to form the straight edge.

The presses described herein can include a cam feature configured to drive the first set of jaws and/or the second set of jaws. In some embodiments, the cam feature can be an external cam connected to the first set of jaws and the second set of jaws by one or more lever arms, but in other embodiments, the cam feature can overlay or be in line with the first and second sets of jaws. The cam feature can include a single cam path or multiple cam paths configured to drive the first set of jaws and the second set of jaws. Where there are multiple cam paths, each cam path can be associated with one of the first set of jaws and one of the second set of jaws.

The press can move between different configurations. The press can include an open configuration where the first set of jaws and the second set of jaws are positioned to allow a tampon blank to be inserted. The press can move between the open configuration and a closed configuration where the first set of jaws and/or the second set of jaws are positioned to shape the elongate body. In the closed position, the innermost surfaces of the first set of jaws can be positioned radially inward relative to the second set of jaws or circumferentially aligned with the innermost surfaces of the second set of jaws. The first set of jaws and the second set of jaws can move in a radial direction between the different positions. The movement of the different sets of jaws can be simultaneous or in an alternating manner. The jaws may generally move along a linear path, for example in a radial direction. There is no non-linear movement of the individual jaws.

The press can be configured to move between the closed configuration and a transfer configuration where the first set of jaws are moved radially outward relative to the second set of jaws. The first set of jaws and the second set of jaws do not move to the transfer configuration at the same time. In the transfer configuration, the second set of jaws can be positioned to maintain the elongate body in the press until the elongate body is ejected from the press. The second set of jaws can shape the elongate body at any point during the press cycle or not at all. The mechanics of the presses described herein are not limited to the specific jaw shapes included here.

Certain aspects of the disclosure are directed toward a method of manufacturing a tampon using any of above described tampon presses. The method can include inserting a tampon blank into a tampon press in an open configuration. The first set of jaws and the second set of jaws move radially inward toward a closed configuration to shape the tampon blank. Shaping the tampon blank can include forming a plurality of wells in the tampon blank using the first set of jaws and/or forming a plurality of longitudinal recesses in the tampon blank using the second set of jaws. The first and second sets of jaws can move simultaneously or in an alternating manner. In the closed configuration the first set of jaws and/or the second set of jaws can form one or more flow paths in the elongate body. The jaws can have any shape and form any flow path described herein. For example, the first set of jaws can form a spiral recess in the elongate body. The method can include moving jaws toward a transfer configuration, for example by moving the first set of jaws radially outward relative to the second set of jaws toward a transfer configuration to eject the shaped tampon blank from the press. The second set of jaws can form a plurality of longitudinal grooves during the closed configuration, transfer configuration, or any intermediate step between the different configurations. After transfer, the first set of jaws and the second set of jaws can move radially outward back to the open configuration.

Depending on the configuration of the press, the first set of jaws and the second set of jaws can be driven by rotating the cam feature in a single direction (e.g., clockwise or counterclockwise) relative to the jaws or in both directions (e.g., clockwise or counterclockwise) relative to the jaws.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1 illustrates a tampon with a longitudinal flow path to direct a flow of menses.

FIG. 2 illustrates a tampon with spiral flow path to direct a flow of menses.

FIG. 3 illustrates a tampon with a right-handed spiral flow path.

FIG. 4 illustrates a tampon with a left-handed spiral flow path.

FIG. 5 illustrates a tampon with a double spiral flow path.

FIGS. 6A-6C illustrate a tampon having a spiral flow path.

FIGS. 7A-7D illustrate a tampon having a spiral flow path and secondary flow paths crossing the spiral flow path.

FIG. 8A illustrates a method of manufacturing a tampon.

FIGS. 8B and 8C illustrate a mold that may be used to manufacture a tampon.

FIG. 9 illustrates another tampon having a spiral flow path.

FIG. 10 illustrates a tampon having latitudinal flow paths.

FIG. 11 illustrates another tampon having latitudinal flow paths.

FIGS. 12A and 12B illustrate a tampon having an internal spiral flow path.

FIGS. 13A and 13B illustrate another tampon having an internal spiral flow path.

FIGS. 14A and 14B illustrate a tampon having a plurality of internal spiral flow paths.

FIGS. 15A and 15B illustrate another tampon having a plurality of internal spiral flow paths.

FIGS. 16A and 16B illustrate a tampon having longitudinal ribs.

FIGS. 17A and 17B illustrate another tampon having longitudinal ribs.

FIGS. 17C and 17D illustrate cross-sections of the tampon shown in FIG. 17A.

FIGS. 18A and 18B illustrate a mold that may be used to manufacture the tampon shown in FIGS. 16A and 16B.

FIGS. 19A to 19B illustrate another tampon having a spiral flow path and a plurality of longitudinal flow paths crossing the spiral flow path.

FIG. 19C is a photograph of a cross-section of a tampon as described with respect to FIGS. 19A to 19B.

FIG. 20A illustrates a partial representation of a tampon press.

FIG. 20B illustrates an enlarged view of the cam in the tampon press in FIG. 20A.

FIGS. 20C-20E illustrate different stages of the tampon press in FIG. 20A.

FIG. 20F illustrates one of the shaping jaws in the tampon press in FIG. 20A.

FIGS. 20G and 20H illustrate partial enlarged views of the projections on the shaping jaw shown in FIG. 20F.

FIG. 201 illustrates one of the grooving jaws in the tampon press in FIG. 20A.

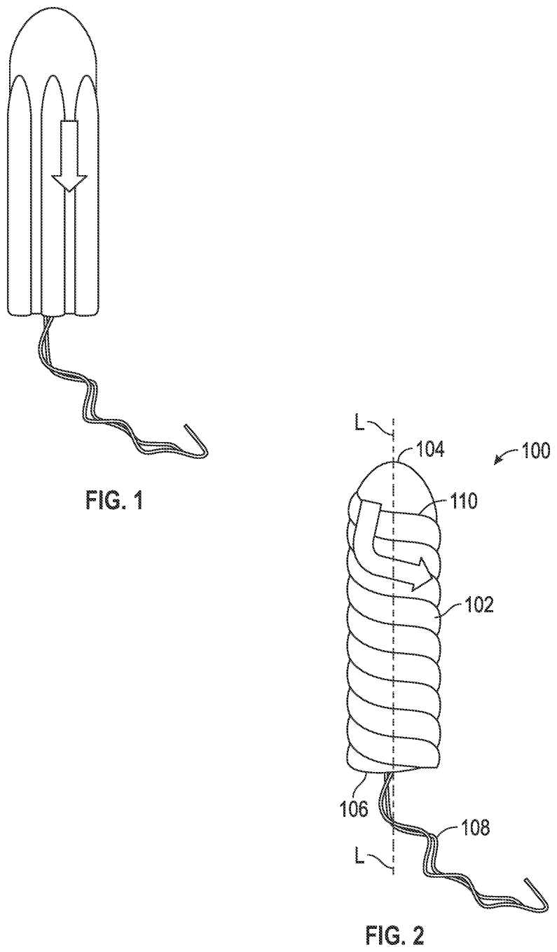

FIG. 21A illustrates another tampon press.

FIG. 21B illustrates the cam in the tampon press in FIG. 21A.

FIGS. 21C-21F illustrate different stages of the tampon press.

FIG. 21G illustrates one of the shaping jaws in the tampon press shown in FIG. 21A.

FIG. 21H illustrates one of the shaping jaws in the tampon press shown in FIG. 21A.

### DETAILED DESCRIPTION

With the absorbent materials and/or flow paths outlined herein, tampons, according to some embodiments, exhibit higher comfort, performance, and sustainability. The tampons described herein, in one embodiment, reduce the likelihood of premature leakage before the absorbent material is fully saturated. The tampons according to several embodiments described herein are particularly advantageous because they offer reduced leakage before full saturation of the tampon to active women, including athletes, who do not have the ability to leave and check their tampon. This allows women to stay engaged and focused without worrying about leakage.

FIG. 2 illustrates a tampon 100 having a body 102 with a first or insertion end 104 and a second or removal end 106. The body 102 may have an elongate shape, for example a generally cylindrical shape. The first end 104 of the elongate body 102 may be rounded and/or tapered to facilitate insertion of the tampon 100. The end face of the first end 104 may have a rounded or flat profile. The first end 104 of the elongate body 102 may have a reduced diameter compared to the second end 106 of the elongate body 102. The second end 106 of the elongate body 102 may include a flattened base. As illustrated, the elongate body 102 has a rounded cone portion and a cylindrical base portion, but in other configurations, the elongate body 102 may be entirely cylindrical.

The length of the elongate body 102, measured along a longitudinal axis L of the elongate body 102, may be greater than a width of the elongate body 102, measured along an axis perpendicular to the longitudinal axis L. For example, the length of the elongate body 102 may be at least three times or at least four times greater than the width of the elongate body 102. The length of the elongate body 102 can be less than or equal to about 7 cm, less than or equal to about 6 cm, or less than or equal to about 5 cm, for example between about 4 cm and about 6 cm. A width of the second end 106 of the elongate body 102 can be less than or equal to about 1.5 cm (e.g., 1.25-1.5 cm, 1.0-1.25 cm, 0.75-1.0 cm and numerical values in between), while a width of the first end 104 of the elongate body 104 can be less than or equal to about 1.0 cm (e.g., 0.75-1.0 cm, 0.5-0.75 cm, 0.25-0.5 cm and numerical values in between). A total mass of the elongate body 102 can be between about 1.5 g and about 2.5 g, for example between about 1.9 g and about 2.1 g.

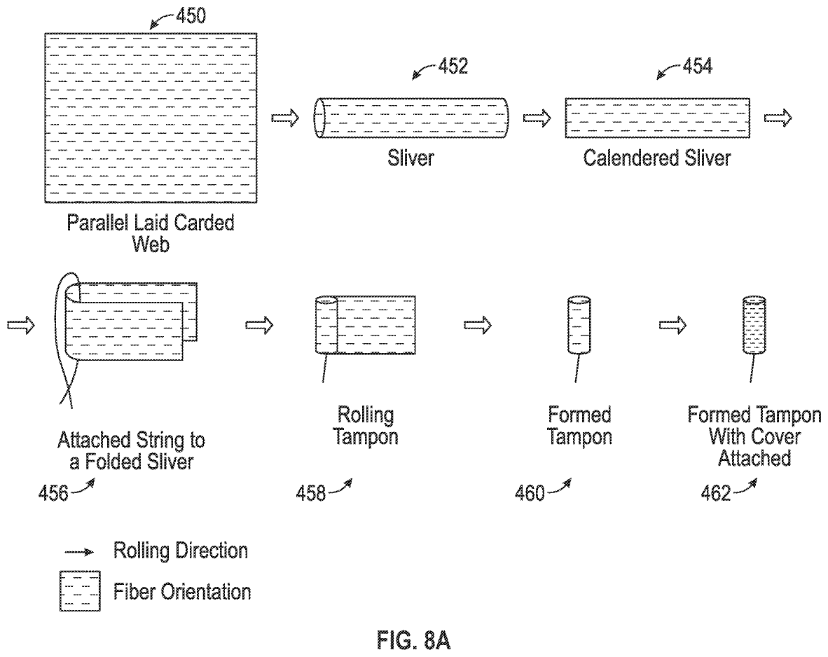

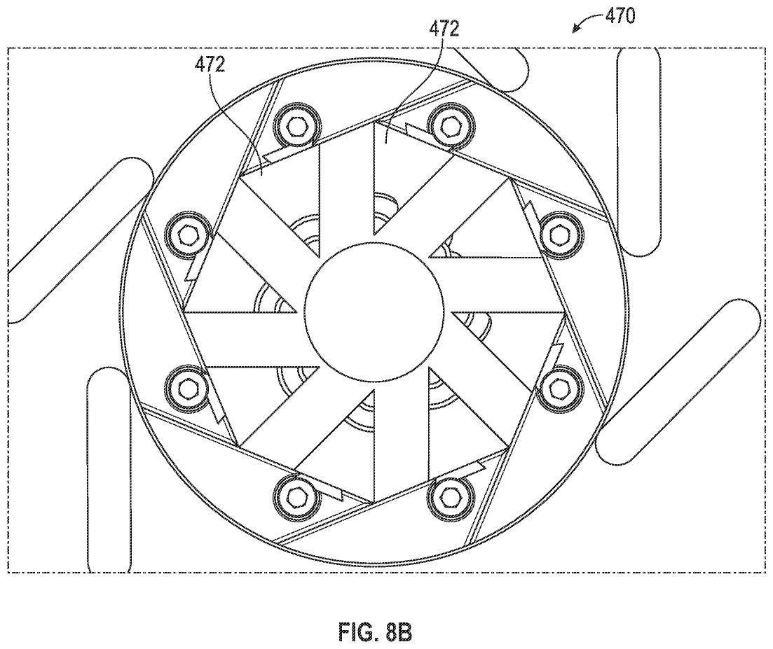

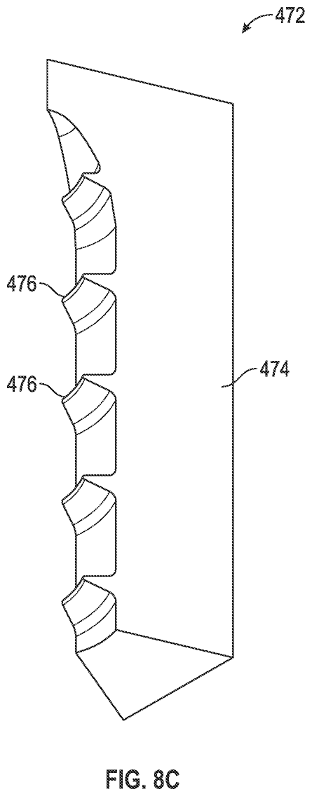

The tampon 100 may also include, optionally, a removal string 108 extending from the second end 106 of the elongate body 102. The removal string 108 facilitates removal of the elongate body 102 without tearing or breaking the tampon 100. The removal string 108 may extend through at least a portion of the elongate body 102. For example, as explained below with respect to FIG. 8A, the removal string 108 may extend along the entire length or substantially the entire length of the elongate body 102. The removal string 108 may be a single fiber or include a plurality of fibers braided together. The removal string 108 may include cotton, polyester, polypropylene, rayon, or a blended material including any of the aforementioned materials. In some embodiments, a string is not included and the tampon may be removed manually for example.

The elongate body 102 includes at least one flow path 110 configured to direct a flow of menses across the elongate body 102. The flow path 110 may at least partially direct a flow of menses in a latitudinal direction. The flow path 110 may be visually distinct from adjacent portions of the elongate body 102. As shown in FIG. 2, the elongate body 102 includes a single, continuous flow path 110 that directs a flow of menses along an external surface of the elongate body 102. However, as described in more detail below, the flow path 110 may direct a flow of menses internally through the elongate body 102 in addition to, or in alternative to, directing flow along the external surface of the elongate body 102. The flow path 110 extends at least one complete turn around a circumference or perimeter of the elongate body 102 or within the elongate body 102. The complete turn of the flow path 110 may include a fully enclosed ring or a 360 degree turn of a spiral. In a spiral configuration, the spiral may include a curve disposed in a single plane or a three-dimensional curve that extends longitudinally along an axis.

The elongate body 102 includes an absorbent material that may have liquid wicking performance. In some configurations, the elongate body 102 may include a liquid permeable cover layer having the same or a different material from the core material of the elongate body 102. The absorbent material may include cotton, organic cotton, rayon, viscose, lyocell, bamboo, foam, or a blend including any of the aforementioned materials. For example, the absorbent material may be a cotton-bamboo blend such as a 50 -50 cotton-bamboo blend. With a cotton-bamboo blend, the bamboo component provides absorbency and antibacterial properties, while the cotton acts to “wick” a saturated product. Bamboo is also a sustainable material. As another example, the absorbent material may be a viscose blend such as a combination of Kelheim Galaxy® fiber and Kelheim Danufil® fiber. The viscose blend may include more Galaxy@ fiber than Danufil® fiber, for example 70% Kelheim Galaxy® fiber and 30% Kelheim Danufil® fiber. The absorbent material may be a blend between viscose fibers and cotton.

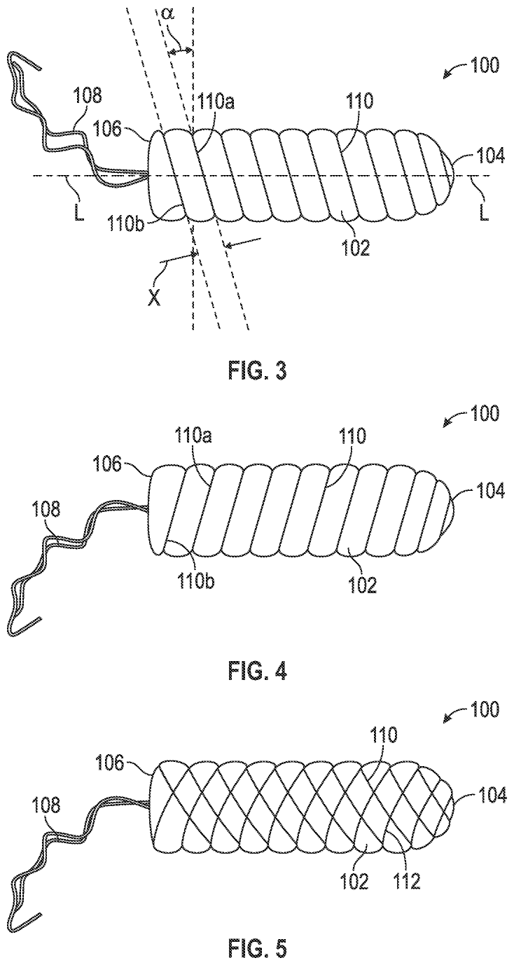

As shown in FIG. 3, the flow path 110 may be a spiral flow path extending across a length of the elongate body 102. As illustrated, the flow path 110 is a right-handed spiral, but the flow path 110 may be a left-handed spiral (see FIG. 4). The flow path 110 begins at or near the first end 104 of the elongate body 102, for example within the rounded or tapered portion of the elongate body 102, and terminates at or near the second end 106 of the elongate body 102. The spiral flow path 110 begins with a free end and terminates at a free end. The spiral flow path 110 may be a single, continuous flow path along an external surface of the elongate body 102 with a single termination point at or near the second end 106 of the elongate body 102. The spiral flow path 110 increases the length of the flow path to provide a slower flow path for menses compared to a longitudinal flow path. The lengthened flow path gives the elongate body more time to absorb the menses and therefore reduces leakage. For a similar sized tampon having the same absorbent material, the spiral flow path increases total time to possible leakage by at least 10% to 20%.

Each turn of spiral flow path 110 rotates about the longitudinal axis L of the elongate body, such that each turn of the spiral flow path 110 is closer to latitudinal than longitudinal when viewed with insertion end 104 vertically above the removal end 102. At least one turn 110a, 110b of the spiral flow path 110 may be disposed at an angle a from horizontal where horizontal is perpendicular to the longitudinal axis L. The angle a can be less than or equal to about 60 degrees, less than or equal to about 40 degrees, or less than or equal to about 20 degrees. Each turn 110a, 110b of the spiral flow path may be disposed at the same angle a or the angles a may vary. Each turn 110a, 100b may be disposed at an angle that is at least about 45 degrees and/or less than or equal to about 90 degrees from the longitudinal axis L, for example between about 45 degrees and about 75 degrees, between about 50 degrees and about 80 degrees, or between about 60 degrees and about 90 degrees. The pitch x between successive turns 110a, 110b of the spiral flow path 110 may be less than or equal to about 20 mm, less than or equal to about 10 mm, or less than or equal to about 5 mm. In some configurations, a distance between any two points disposed 360 degrees apart along the spiral flow path 110 may be constant.

The spiral flow path 110 continually travels in a single or longitudinal direction from a single starting point at or near the insertion end 104 to a single terminating point at or near the removal end 106. When moving from the starting point to the terminating point, any selected location along the spiral flow path is closer to the removal end 106 of the elongate body 102 than any previous location between the starting point and the selected location. As the spiral flow path 110 travels toward the removal end 106, the spiral flow path 110 never turns back toward insertion end 104. This enables the menses to flow in a direction that is closer to latitudinal than longitudinal at any given segment of the flow path 110.

As explained above, the elongate body 102 may include more than one flow path. For example, as shown in FIG. 5, the elongate body 102 may include a first, right-handed spiral flow path 110 twisted together with a second, left-handed spiral flow path 112.

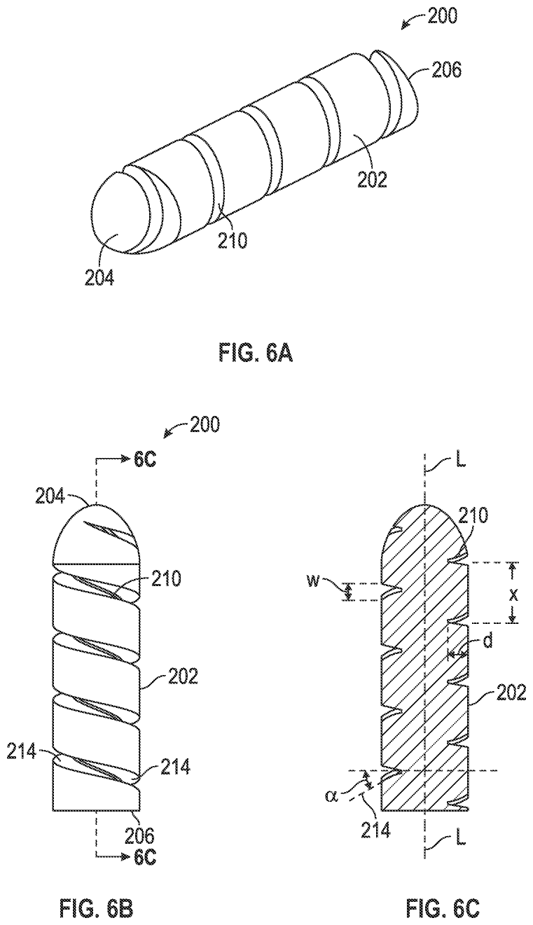

FIGS. 6A-6C illustrate a tampon 200 having an elongate body 202 with a first or insertion end 204 and a second or removal end 206. The elongate body 202 may include any of the absorbent materials described above. Although not shown, the tampon 200 may include a removal string extending from the removal end 206 of the elongate body 202. The tampon 200 may include any of the features described above with respect to the tampon 100.

The tampon 200 includes a spiral flow path 210 configured to direct a flow of menses along an outer surface of the elongate body 202. The spiral flow path 210 may be a groove, channel, or indentation that provides a recess in the outer surface of the elongate body 202. The spiral flow path 210 begins at or near the first end 204 of the elongate body 202 (see FIG. 6B) and terminates at or near the second end 206 of the elongate body 202 (see FIG. 6A). The spiral flow path 210 may provide a single, continuous recess terminating at a single location at the second end 206 of the elongate body 202. As shown in FIGS. 6A and 6B, there is only one spiral flow path 210 without any additional grooves, channels, indentations, or other flow paths.

When the tampon 200 sits within the vaginal cavity, the spiral flow path 210 enables the tampon to conform to the rugae along the vaginal walls to reduce the likelihood of displacement. For a 2.0 g tampon having a spiral flow path 210, the tampon 200 exhibits a tampon absorption rate of at least 9 g and has super absorbency rating according to the Syngyna test outlined by the Food and Drug Administration under 21 C.F.R. § 801.430 (“User labeling for menstrual tampons”). This means the tampon absorbed at least 9 g of fluid prior to the first drop of fluid leakage. Tampons having a spiral flow path 210 absorb at least about 3.75 grams of fluid per gram of dry absorbent material in the tampon, at least about 4.0 grams of fluid per gram of dry absorbent material in the tampon, or at least 4.25 grams of fluid per gram of dry absorbent material in the tampon. For example, tampons having the spiral flow path may absorb between about 4.0 grams to about 4.5 grams of fluid per gram of dry absorbent material according to the Syngyna test.

The spiral flow path 210 extends at least one complete turn around a circumference or perimeter of the elongate body 202. For example, the spiral flow path 210 may include at least about two complete turns (e.g., at least about three complete turns) and/or less than or equal to about ten complete turns, for example between about two complete turns and about four complete turns or between about three complete turns and about five complete turns. As shown in FIGS. 6A and 6B, the spiral flow path 210 extends about four complete turns around the circumference of the elongate body 202. However, the spiral flow path 210 may include a fewer or greater number of turns depending on the pitch x between successive turns of the spiral flow path 210. The pitch x may be less than or equal to about 20 mm, less than or equal to about 10 mm, or less than or equal to about 5 mm (see FIG. 6C). In some embodiments, the pitch x is between about 7.5 mm and about 10 mm or between about 5 mm and about 7.5 mm. The pitch x may be at least about 10% of a length of the elongate body 202 and/or less than or equal to about 40% of the length of the elongate body 202. In some embodiment, the pitch x is between about 10% and about 15% of a length of the elongate body 202 or between about 15% and about 20% of a length of the elongate body 202. The pitch x may be constant or varied along the length of the spiral flow path 210.

A depth d of the spiral flow path 210, measured from an outer surface of the elongate body 202 to an apex of the spiral flow path 210, is sufficiently shallow to maintain the integrity of the elongate body 202. For example, the depth d may be less than or equal to about 5 mm, for example less than or equal to about 3 mm. The depth d of the recess may be less than or equal to about 40% (e.g., 30%-40%, 20%-30%, 15%-25% and numerical values in between) of a width of the elongate body 202, for example less than or equal to about 25% (e.g., 20%-25%, 15%-20%, 10%-15% and numerical values in between) of a width of the elongate body 202. The depth d of the recess may be constant or varied along the length of the spiral flow path 210.

The width w at an opening of the spiral flow path 210 may be less than or equal to about 5 mm, for example less than or equal to about 2 mm. In some embodiments, the width is between about 1.5 mm and about 2.5 mm (e.g., 1.5-2.0 mm, 1.75-2.25 mm and numerical values within those ranges). The width w may be less than or equal to about 15% of the length of the elongate body 202, for example less than or equal to about 10% of the length of the elongate body 202 (e.g., 10% to 15%, 7.5% to 12.5%, 5% to 10% and numerical values within those ranges). The width w at the opening of the spiral flow path 210 may be constant or varied along the length of the spiral flow path 210.

The spiral flow path 210 includes an inner surface 214. As illustrated, the spiral flow path 210 includes two inner surfaces 214 tapered toward an apex of the recess. However, in other configurations, the width w at the opening of the spiral flow path 210 may be the same or narrower than the opposite side of the spiral recess (e.g., at the most radially inward edge of the spiral flow path 210). For example, the two inner surfaces 214 may be parallel or diverge from each other. In other configurations, the inner surface 214 of the flow path 210 may be rounded such that the inner surface 214 has a concave profile when viewed in cross-section. The radius of curvature at the open side of the flow path 210 may be greater than, less than, or the same as the radius of curvature at the innermost edge of the flow path 210. The inner surface 214 and the outer surface of the elongate body 202 may form a sharp edge or a rounded edge. An inner surface 214 of the spiral flow path 210 may be disposed at an angle a from horizontal, measured perpendicular to the longitudinal axis L of the elongate body 202 (see FIG. 6C). The angle a may be less than or equal to about 40 degrees, less than or equal to about 30 degrees, less than or equal to about 20 degrees, less than or equal to about 15 degrees, or less than or equal to about 10 degrees. In some embodiments, the angle a is between 30 degrees and about 40 degrees, between about 25 degrees and about 35 degrees, between about 15 degrees and about 25 degrees and numerical values within those ranges. The angle a may be constant or varied along the length of the spiral flow path 210. Each turn of the spiral flow path 210 may be disposed at an angle that is between about 45 degrees and about 90 degrees from the longitudinal axis L, for example between about 45 degrees and about 75 degrees, between about 50 degrees and about 80 degrees, or between about 60 degrees and about 90 degrees.

Each turn of the spiral flow path 210 may have a radius of curvature of less than or equal to about 10 mm, less than or equal to about 8 mm, or less than or equal to about 6 mm (e.g., 2-6 mm, 3-5 mm, 4-8 mm and numerical values within those ranges). The radius of curvature may be constant or varied along the length of the spiral flow path 210. The entire spiral flow path 210 may have a curvature without any corners or apexes forming a point.

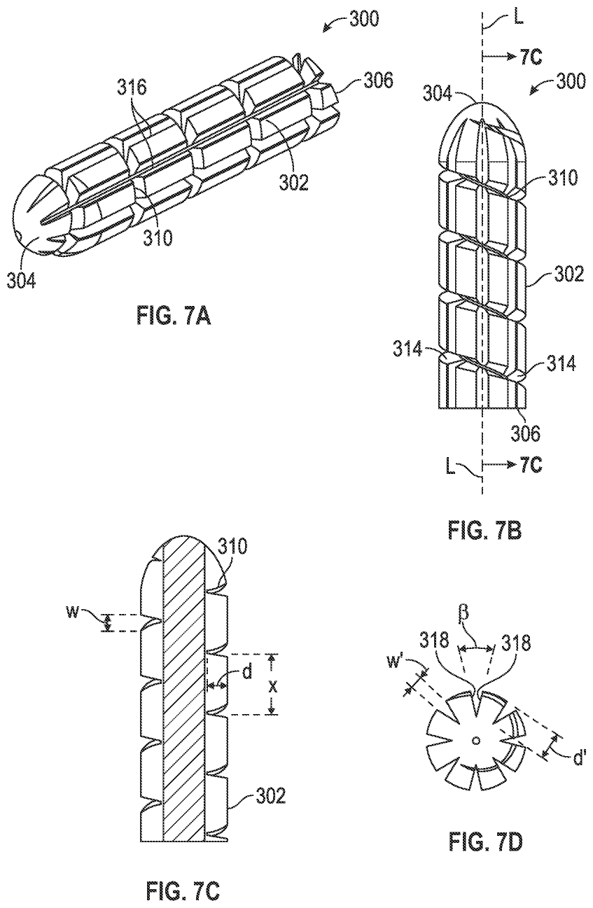

With reference to FIGS. 7A-7D, another tampon 300 is shown. The tampon 300 resembles or is identical to the tampon 200 except as described below.

In addition to the spiral flow path 310, the elongate body 302 may have one or more secondary flow paths 316. For example, the elongate body 302 may include a plurality of secondary flow paths 316 spaced apart from each other around a circumference of the elongate body 302. Each secondary flow path 316 may cross successive turns of the spiral flow path 310. Each secondary flow path 316 may extend in a generally longitudinal direction or at least closer to the longitudinal direction than the turns of the spiral flow path 316. The secondary flow paths 316 facilitate tampon expansion, which can facilitate more even absorption. An under-expanded tampon may lead to premature leakage.

The secondary flow paths 316 may be grooves, channels, or indentations that form a recess in an outer surface of the elongate body 302. Each secondary flow path 316 may begin at or near the first end 304 of the elongate body 302 and terminate at or near the second end 306 of the elongate body 302. Each secondary flow path 316 may be circumferentially spaced apart by at least about 2 mm, at least about 4 mm, or at least about 6 mm (e.g. 2-4 mm, 3-5 mm, 4-6 mm and numerical values within those ranges). The space between successive secondary flow paths 316 may be constant or varied around the circumference of the elongate body 302.

A depth d′ of the secondary flow path 316, measured from the outer surface of the elongate body 302 to an apex of the secondary flow path 316, may be the same or different from the depth d of the spiral flow path 310. For example, the depth d′ may be less than or equal to about 5 mm or less than or equal to about 3 mm (e.g., 2-4 mm, 2.5-4.5 mm, 3-5 mm and numerical values within those ranges). The depth d′ of the secondary flow path 316 may be less than or equal to about 40% of a width of the elongate body 302, for example less than or equal to about 25% of a width of the elongate body 302 (e.g., 15% to 25%, 20% to 30%, 25% to 35% and numerical values within those ranges). The depth d of the secondary flow path 316 may be constant or varied along a length of the spiral flow path 310.

The width w′ at an opening of the secondary flow path 316 may be the same or different from the width w of the spiral flow path 310. For example, the width w′ may be less than or equal to about 5 mm (e.g., 3-5 mm, 1-3 mm, 0.5-2 mm and numerical values within those ranges) or less than or equal to about 2 mm. The width w′ may be less than or equal to about 10% (e.g., 8-10%, 6-8%, 3-6%, 1-5%, and numerical values within those ranges) of a circumference of the elongate body 302, for example less than or equal to about 5% of a circumference of the elongate body 302. The width w′ at the opening of the secondary flow path 316 may be constant or varied along the length of the spiral flow path 310.

Each secondary flow path 316 may be defined by adjacent inner surfaces 318 (see FIG. 7D). An angle ß between the adjacent inner surfaces 318 may be less than or equal to about 40 degrees, less than or equal to about 30 degrees, less than or equal to about 20 degrees, less than or equal to about 15 degrees, or less than or equal to about 10 degrees. For example, the angle β may be between about 20 degrees and 25 degrees, between about 17.5 and 22.5 degrees, between about 15 degrees and 20 degrees and numerical values within those ranges. The angle β may be constant or varied along the length of the spiral flow path 310.

FIG. 8A illustrates a method of manufacturing the tampons 100, 200, 300. Fibers of any material described herein can be carded into a web, for example a parallel laid web in which the fibers are oriented parallel to each other (step 450). The basis weight of the web may be at least about 20 g/m² and/or less than or equal to about 50g/m², for example between about 20 g/m² and about 40 g/m² (e.g., 20-30 g/m², 25-35 g/m², 30-40 g/m² and numerical values within those ranges). After the web is formed, the web is passed through a can coiler sliver former (step 452). The sliver may then be densified using a cold calendaring process (step 454). A single tampon may include about 1.5 g to about 2.5 g of the calendared sliver, for example between about 1.9 g and 2.1 g. The calendared sliver may be folded so that a removal string may be applied to the sliver (step 456). The folded sliver may be rolled, for example in a swiss roll design, in the same direction as the orientation of the fibers (step 458) to form the tampon shown in step 460. The horizontally oriented fibers slow fluid flow in the longitudinal direction. In some embodiments, the tampon may be rolled such that a central core of the elongate body is more dense than an outer portion of the elongate body. In other embodiments, the density of the elongate body may be constant throughout.

Optionally, a cover layer may be provided over the elongate body (step 462). The cover layer may be a layer of nonwoven material, which may be the same or different from the core material. The cover layer may be a layer of carded, nonwoven material or a spunbound material. The cover layer may have a lower basis weight than the core. For example, the basis weight of the cover layer may be less than or equal to about 20 g/m² or less than or equal to about 15 g/m² (e.g., 7.5-12.5 g/m², 10-15 g/m², 12.5-17.5 g/m² and numerical values within those ranges). The cover layer may be bonded to the absorbent core of the elongate body, for example thermally bonded using a hot knife.

Although a particular manufacturing method is described with respect to FIG. 8A, the tampons described herein may be manufactured using other methods. For example, after the elongate body is formed, the elongate body may be twisted to form the external spiral flow path having at least one complete turn.

As described above, the elongate body may include one or more flow paths in the outer surface of the elongate body. The flow paths may be molded into the outer surface of the elongate body, for example using a radial compression mold. The mold forms grooves, channels, or indentations that form recesses in the outer surface of the elongate body. The localized compression along the flow path adds more surface area and friction as fluid meets the flow path, thereby slowing the rate of fluid flow in the longitudinal direction. The slower fluid flow provides more time for the elongate body to absorb fluid, promotes full saturation of the tampon, and delays possible leakage. FIG. 8B provides an example of a compression mold 470 including one or more mold segments 472 disposed circumferentially around the mold 470. Each mold segment 472 may be tapered toward a center of the mold 470. Each segment 472 may span between 30 degrees and 180 degrees of the elongate body, for example between 45 degrees and 90 degrees of the elongate body. In use, the formed tampon may be disposed at the center of the one or more mold segments 472. As the one or more mold segments 472 move radially inward toward the elongate body of the tampon, the flow path is formed in the outer surface of the elongate body. This compression process may be manual or automated. FIG. 8C illustrates a single mold segment 472. As illustrated, the mold segment 472 includes a body portion 474 and one or more projections 476 projecting from the body portion 474. The one or more projections 476 form a negative of at least a partial segment of the flow path to be created on the elongate body. Thus, the projections 476 may have dimensions similar to those described with respect to the flow path 210 above. When all the mold segments 472 are combined, there is a complete negative of the flow path to be created on the elongate body.

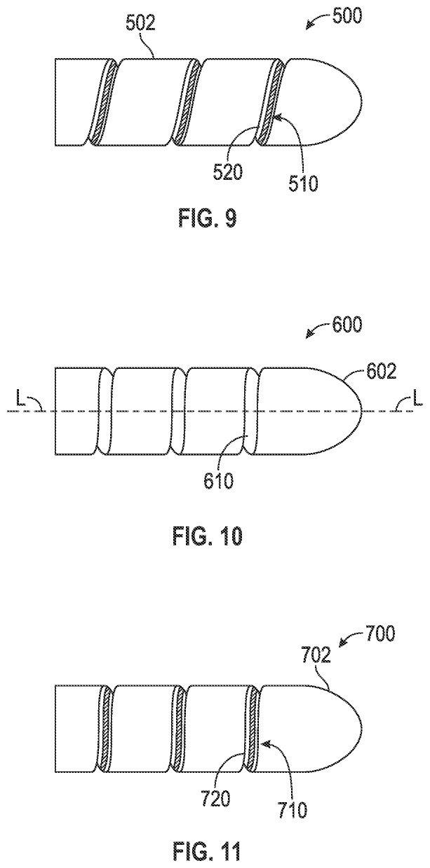

FIG. 9 illustrates a tampon 500 that resembles or is identical to tampons 200, 300 except as described below. As shown in FIG. 9, the elongate body 502 includes a spiral flow path 510 similar to the spiral flow paths 210, 310. In this configuration, the elongate body 502 includes a thread 520 extending around the elongate body 502 to form the spiral flow path 510. The thread 520 compresses the outer surface of the elongate body 502 to form a spiral recess in the outer surface of the elongate body 502. The thread 520 may include a same or different material from the absorbent material of the elongate body 502 or the removal string. For example, the thread 520 may include cotton, polyester, polypropylene, or a blend including any of the aforementioned materials. A hydrophilic or hydrophobic coating may be applied to the thread 520. The thread 520 may be a single fiber or braided from multiple fibers. The thread 520 may be a filament, ribbon, wire, or any other string-like structure.

The elongate body 502 of the tampon 500 may be formed using the method shown in FIG. 8A. After the elongate body 502 is formed, the thread 520 may be fastened around the elongate body 502 to compress the outer surface of the elongate body 502 and form the spiral flow path 510. The thread 520 may be woven into the spiral orientation, for example using a rotating tool. The thread 520 may be chemically, thermally, or mechanically bonded to the outer surface of the elongate body 502. At least the ends of the thread 520 may be bonded to the elongate body 502. In some methods, the thread 520 may be bonded to the elongate body 502 along an entire length of the thread 520.

FIG. 10 illustrates a tampon 600 that may include any features of the above-described tampons. The tampon 600 includes an elongate body 602 and one or more flow paths 610. Each of the one or more flow paths 610 may be a groove, channel, or indentation that forms a recess in an outer surface of the elongate body 602. Unlike the spiral configurations described above, each flow path 610 may form a fully enclosed ring, such as an annular ring disposed in a single plane. For example, as shown in FIG. 10, each flow path 610 may be disposed in a single plane that is perpendicular to the longitudinal axis L of the elongate body 602. However, in other configurations, each flow path 610 may be disposed at an oblique angle relative to the longitudinal axis L, for example at an angle that is less than or equal to about 60 degrees, less than or equal to about 40 degrees, or less than or equal to about 20 degrees relative to the longitudinal axis L (e.g., 50-60 degrees, 45-55 degrees, 40-50 degrees, 35-45 degrees and numerical values within those ranges). In some configurations, the flow path 610 may be disposed at an angle that is between about 45 degrees and about 90 degrees from the longitudinal axis L, for example between about 45 degrees and about 75 degrees, between about 50 degrees and about 80 degrees, or between about 60 degrees and about 90 degrees.

The elongate body 602 may include at least two flow paths 610 and/or less than or equal to ten flow paths 610, for example between three flow paths 610 and five flow paths 610. However, the elongate body 602 may include a fewer or greater number of flow paths 610 depending on the distance between successive flow paths 610. Successive flow paths 610 may be separated by a distance of less than or equal to about 20 mm, less than or equal to about 10 mm, or less than or equal to about 5 mm. In some embodiments, the distance is between about 7.5 mm and 10 mm or between about 5 mm and about 7.5 mm. The distance between successive flow paths 610 may be at least about 10% of a length of the elongate body 602 and/or less than or equal to about 40% of a length of the elongate body 602. In some embodiment, the distance is between about 10% and 15% of a length of the elongate body 602 or between about 15% and about 20% of a length of the elongate body 602. The distance between successive flow paths 610 may be constant or varied along a length of the elongate body 602.

FIG. 11 illustrates a tampon 700 that resembles or is identical to the tampon 600 except as described below. As shown in FIG. 11, the elongate body 702 includes a plurality of latitudinal flow paths 710 similar to the flow paths 610. In this configuration, the elongate body 702 includes a plurality of threads 720 extending around the elongate body 702 to form the one or more flow paths 710. The plurality of threads 720 compress the outer surface of the elongate body 702 to form the recessed flow paths 710.

For a 2.0 g tampon having the latitudinal flow paths 610, 710, the tampons 600, 700 exhibit a tampon absorption rate of at least 9 g and has super absorbency rating according to the Syngyna test. This means the tampon absorb at least 9 g of fluid prior to the first drop of fluid leakage. Tampons 600, 700 having a plurality of latitudinal flow paths 610, 710 absorb at least about 3.75 grams of fluid per gram of dry absorbent material in the tampon or at least about 4.0 grams of fluid per gram of dry absorbent material in the tampon. For example, tampons 600, 700 having a plurality of latitudinal flow paths 610, 710 may absorb between about 3.75 grams and about 4.0 grams or between about 4.0 grams and about 4.25 grams of fluid per gram of dry absorbent material according to the Syngyna test. For a similar sized tampon having the same absorbent material, the latitudinal flow paths 610, 710 increase total time to possible leakage by at least 5% to 35%.

As mentioned above, in addition, or in alternative to, the one or more flow paths disposed in the outer surface of the elongate body, the tampon may include one or more flow paths disposed within or internal of the elongate body to direct a flow of menses through the elongate body. The internal flow paths are not visible from an exterior of the tampon. The internal flow paths lengthen the total flow path, thus providing more time for the elongate body to absorb the menses and reducing the likelihood of leakage.

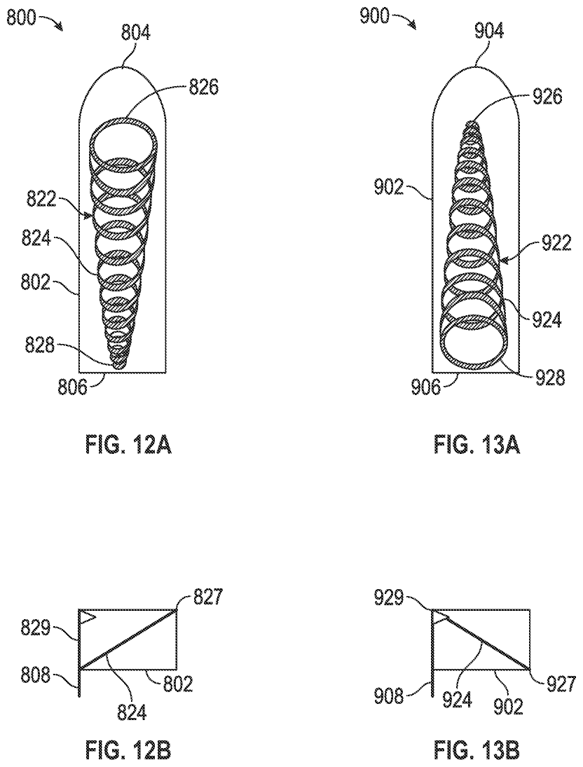

For example, as shown in FIG. 12A, the tampon 800 includes an internal flow path 822 positioned within the elongate body 802. The internal flow path 822 may provide a single, continuous spiral along at least a partial length or substantially the entire length of the elongate body 802. The internal flow path 822 includes a first end 826 positioned at or near the first end 804 of the elongate body 802 and a second end 828 that terminates at or near the second end 806 of the elongate body 802. As shown in FIG. 12A, there is only one internal flow path 822 without any additional internal flow paths.

The internal flow path 822 may include any of the features described above with respect to the spiral flow path 210. The internal flow path 822 extends at least one complete turn within the elongate body 802. Successive turns of the internal flow path 822 may have the same or different radius. As shown in FIG. 12A, the radius of the turns varies along a length of the internal flow path 822. A first radius of a turn at the first end 826 of the internal flow path 822 may be greater than a second radius of a turn at the second end 828 of the internal flow path 822.

The internal flow path 822 may be formed by a thread 824 extending through the elongate body 802. The thread 824 may include any of the properties of the thread 520 described above with respect to the tampon 500. The thread 824 may be continuous with or separate from the removal string 808.

The elongate body 802 may be formed using a similar method to that shown in FIG. 8A. However, after the removal string is provided (see step 456), the thread 824 is provided to a surface of the sliver that will be internal of the elongate body 802. As shown in FIG. 12B, a single thread 824 is positioned diagonally across the folded sliver. The thread 824 may be bonded to the folded sliver or freely positioned on the folded sliver. The thread 824 may extend from a first location at or near a first corner 829, formed by the folded edge of the sliver and the edge that will form the second end 806 of the elongate body 802, toward a second location at or near a second corner 827, formed by the free edges of the sliver and the edge that will form the first end 804 of the elongate body 802. After the thread 824 is provided, the folded sliver may be rolled in the direction of the arrow, similar to step 458 of FIG. 8A.

FIG. 13A illustrates a tampon 900 that resembles or is identical to the tampon 800 except as described below. As shown in FIG. 13A, the internal flow path 922 includes a first end 926 positioned at or near the first end 904 of the elongate body 902 and a second end 928 positioned at or near the second end 906 of the elongate body 902. A first radius of a turn at the first end 926 of the internal flow path 922 may be smaller than a second radius of a turn at the second end 928 of the internal flow path 922.

The tampon 900 may be formed using the same method described above with respect to the tampon 800 except that the thread 924 is applied to the folded sliver in the opposite direction. As shown in FIG. 13B, a single thread 924 is positioned diagonally across the folded sliver. The thread 924 may extend from a first location at or near a first corner 929, formed by the folded edge of the sliver and the edge that will form the first end 904 of the elongate body 902, toward a second location at or near a second corner 927, formed by the free edges of the sliver and the edge that will form the second end 906 of the elongate body 902. The thread 924 may be continuous with or separate from the removal string 908.

For a 2.0 g tampon having the internal flow path 822, 922, the tampon 800, 900 exhibits a tampon absorption rate of at least 8.5 g, or at least 9.0 g, according to the Syngyna test. Tampons 800, 900 having the internal flow path 822, 922 absorb at least about 3.75 grams of fluid per gram of dry absorbent material in the tampon or at least about 4.0 grams of fluid per gram of dry absorbent material in the tampon. For example, tampons 800, 900 having an internal flow path 822, 922 may absorb between about 3.75 grams and about 4.0 grams or between about 4.0 grams and about 4.25 grams of fluid per gram of dry absorbent material according to the Syngyna test. For a similar sized tampon having the same absorbent material, the internal flow path 822, 922 increases total time to possible leakage by at least 5% to 25%.

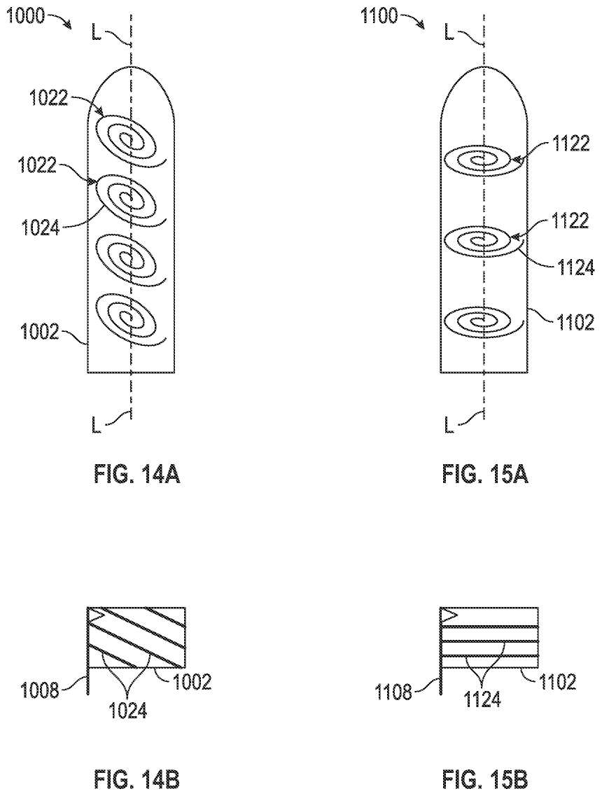

FIGS. 14A and 14B illustrate a tampon 1000 with a different internal flow path configuration. As shown in FIG. 14A, the tampon 1000 includes an elongate body 1002 with one or more internal flow paths 1022 positioned within the elongate body 1002.

Each flow path 1022 may be disposed within a single plane and at an oblique angle relative to a longitudinal axis L of elongate body 1002. As shown in FIG. 14A, the elongate body 1002 includes a plurality of internal flow paths 1022 longitudinally spaced apart along a length of the elongate body 1002. For example, the elongate body 1002 may include at least two internal flow paths 1022 and/or less than or equal to ten internal flow paths 1022, for example between three internal flow paths 1022 and five internal flow paths 1022. Successive internal flow paths 1022 may be separated by a distance that is less than or equal to about 30 mm, less than or equal to about 20 mm, less than or equal to about 10 mm, or less than or equal to about 5 mm. In some embodiments, the distance is between about 7.5 mm and about 10 mm or between about 5 mm and about 7.5 mm. The distance between successive internal flow paths 1022 may be at least about 10% of a length of the elongate body 1002 and/or less than or equal to about 40% of a length of the elongate body 202. In some embodiment, the distance is between about 10% and about 15% of a length of the elongate body 1002 or between about 15% and about 20% of a length of the elongate body 1002. The distance between successive internal flow paths 1022 may be constant or varied along a length of the elongate body 1002.

Each internal flow path 1022 may be disposed at a same or different angle relative to the longitudinal axis L of the elongate body 1022. For example, each internal flow path 1022 may be disposed relative to the longitudinal axis L at an angle 0 that is less than or equal to about 60 degrees, for example between about 20 degrees and about 45 degrees (e.g., 20-30 degrees, 25-35 degrees, 30-40 degrees and numerical values within those ranges). Successive flow paths 1022 may be parallel to each other.

Each of the internal flow paths 1022 may be formed by a thread 1024 spiraling around a single point within the elongate body 1002. The spiraling thread includes at least one complete turn. The threads 1024 may include any of the properties of the thread 520 described above with respect to the tampon 500.

The elongate body 1002 may be formed using a similar method to that shown in FIG. 8A. However, after the removal string is provided (see step 456) one or more threads 1024 are provided to a surface of the sliver that will be internal of the elongate body 1002. As shown in FIG. 14B, a plurality of threads 1024 are positioned diagonally across the folded sliver and parallel to each other. Each thread 1024 begins at a first location closer to the edge of the sliver that will form the first end 1004 of the elongate body 1002 than the second end 1006 of the elongate body 1002 and terminates at a second location closer to the edge of the sliver that will form the second end 1006 of the elongate body 1002 than the first end 1004 of the elongate body 1002. The threads 1024 are provided at an angle that is dependent on the desired angle 0 of the flow path 1022 relative to the longitudinal axis L of the elongate body 1002. The threads 1024 may be bonded to the folded sliver or freely positioned on the folded sliver. After the threads 1024 are provided, the folded sliver may be rolled in the direction of the arrow, similar to step 458 of FIG. 8A.

Although not shown, the flow paths 1022 may be angled in the opposite direction. In the opposite configuration, each thread 1024 begins at a first location closer to the edge of the sliver that will form the second end 1006 of the elongate body 1002 than the first end 1004 of the elongate body 1002 and terminates at a second location closer to the edge of the sliver that will form the first end 1004 of the elongate body 1002 than the second end 1006 of the elongate body 1002.

FIG. 15A illustrates a tampon 1100 that resembles or is identical to the tampon 1000 except as described below. As shown in FIG. 15A, each of the plurality of internal flow paths 1122 lies in a plane perpendicular to the longitudinal axis L. The tampon 1100 may be formed using the same method described above with respect to the tampon 1000 except that the one or more threads 1124 are applied to the folded sliver in a latitudinal direction. The plurality of threads 1124 may be provided in the same direction as the fibers of the carded web.

For a 2.0 g tampon having the internal flow path 1022, 1122, the tampon 1000, 1100 absorb at least about 3.5 grams of fluid per gram of dry absorbent material in the tampon or at least about 3.75 grams of fluid per gram of dry absorbent material in the tampon. For example, tampons 1000, 1100 having internal flow paths 1022, 1122 may absorb between about 3.5 grams and about 3.75 grams or between about 3.75 grams and about 4.0 grams of fluid per gram of dry absorbent material according to the Syngyna test. For a similar sized tampon having the same absorbent material, the internal flow paths 1022, 1122 increases total time to possible leakage by at least 5% to 15%.

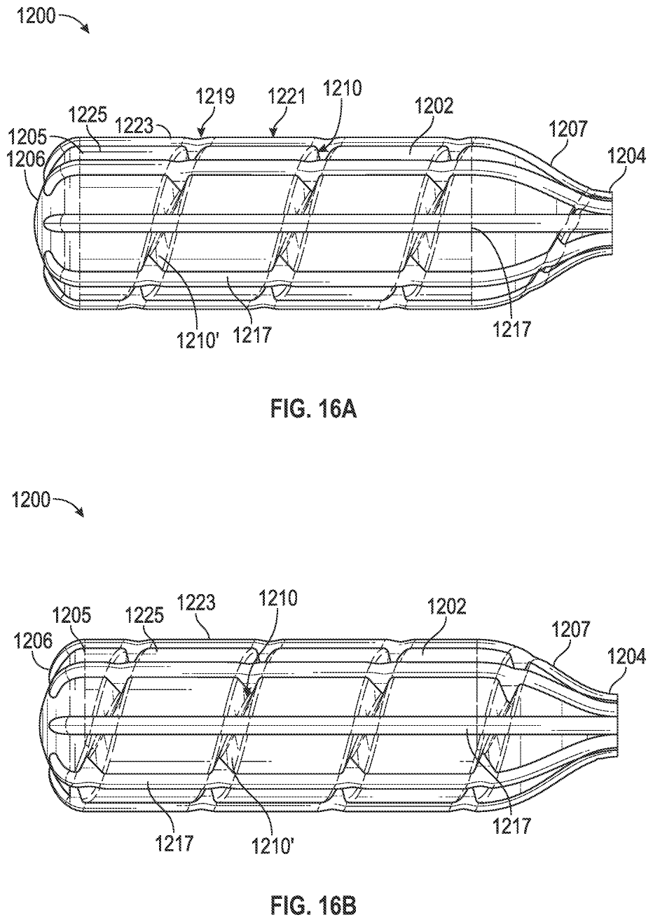

FIGS. 16A and 16B illustrate front and rear views of another tampon 1200. The elongate body 1202 may include one or more longitudinal ribs 1217 crossing a flow path 1210. The flow path 1210 may include any of the features described above with respect to the flow path 210. For example, the flow path 1210 may be shaped to redirect fluid at least partially in a horizontal direction to slow the time it takes for the fluid to reach the second end 1206 of the tampon and subsequently leak. The flow path 1210 may include a spiral shape to redirect fluid around a circumference of the elongate body 1202 rather than allowing the fluid to flow straight down from the first end 1204 to the second end 1206 of the elongate body 1202. The flow path 1210 may be continuous about the elongate body 1202 of the tampon 1200 except for the longitudinal ribs 1217. In some embodiments, the tampon 1200 only has a spiral, recessed flow path 1210 and longitudinal ribs 1217 with no other recessed flow paths or ribs extending in other directions. But in other embodiments, the longitudinal ribs 1217 may provide alone or in combination with any shape or number of flow paths.

The direction of fluid flow along flow path 1210 may be dependent on at least the shape and orientation of the flow path 1210. In some embodiments, fluid that enters the flow path 1210 may flow generally unidirectionally along the flow path 1210 and toward the second end 1206. In other embodiments, fluid that enters the flow path 1210 may flow in more than one direction along the flow path 1210. For example, fluid may flow bidirectionally along the flow path 1210. For a spiral flow path 1210, fluid may flow in both the clockwise and counterclockwise direction along the flow path 1210.

Fluid that approaches the elongate body 1202 from the first end 1204 may enter the flow path 1210 at or near an entry end of the flow path 1210. At least some excess fluid may bypass at least a portion of the flow path 1210 and flow in a longitudinal direction toward the second end 1206. The excess fluid may enter the flow path 1210 at a later portion of the flow path 1210, for example beyond the entry end or first turn of the flow path 1210. The excess fluid that enters at a later portion of the flow path 1210 may travel unidirectionally or bidirectionally along the flow path 1210 depending on the shape and orientation of the flow path 1210. Whether unidirectional or bidirectional, flow along the flow path 1210 may be non-vertical or closer to horizontal compared to flow in the longitudinal direction. This slows the time it takes for the fluid to reach the second end 1206.

The elongate body 1202 has an outer surface 1225. The longitudinal ribs 1217 protrude from the outer surface 1225, while the flow path 1210 is recessed from the outer surface 1225. The outer surface 1225 may extend longitudinally between successive turns 1210 of the flow path 1210 and/or circumferentially between adjacent ribs 1217. Sections of the flow path 1210 may be positioned between adjacent ribs 1217. Each section of the flow path 1210 may be non-parallel to the adjacent ribs 1217. The sections of the flow path 1210 may extend at an oblique angle relative to the ribs 1217.

The longitudinal ribs 1217 maximize tampon expansion when saturated, which prevents premature leakage caused by an under-expanded tampon. The longitudinal ribs 1217 may have a fibrous construction that quickly expands and relaxes upon release, such that the longitudinal ribs 1217 do not substantially affect fluid flow through the flow path 1210. When the tampon 1200 comes into contact with fluid, the fluid can follow the flow path 1210 substantially uninterrupted. The longitudinal ribs 1217 do not obstruct the slowing effects of the flow path 1210.

The plurality of longitudinal ribs 1217 may be spaced apart from each other around a circumference of the elongate body 1202. The tampon 1200 may include at least two longitudinal ribs 1217, at least four longitudinal ribs 1217, at least six longitudinal ribs 1217, or at least eight longitudinal ribs 1217. Each longitudinal rib 1217 may extend in a generally longitudinal direction or at least closer to the longitudinal direction than the turns of the flow path 1210. As illustrated, the longitudinal ribs 1217 are generally straight and aligned with the longitudinal axis of the elongate body, but in other configurations may include ribs that are angled, waved, curved, or otherwise shaped relative to the longitudinal axis of the elongate body.

The flow path 1210 may be a continuous spiral flow path intersected by the longitudinal ribs 1217. Each longitudinal rib 1217 may extend into the flow path 1210 at locations 1219 where the longitudinal rib 1217 crosses the flow path 1210. Viewed another way, the elongate body 1202 may include a plurality of flow path segments 1210′ separated by the longitudinal ribs 1217. The plurality of flow path segments 1210′ generally direct fluid in a spiral flow path around the elongate body 1202. Circumferentially adjacent flow path segments 1210′ may be separated by one of the ribs 1217. The flow path segments 1210′ between any two adjacent ribs 1217 may be longitudinally spaced apart from each other.

Each longitudinal rib 1217 may be integral with or joined to a recessed surface of the flow path 1210. The outer edge 1223 of the longitudinal rib 1217 may extend radially outward of an outer surface 1225 of the elongate body 1202 along a majority of or substantially the entire length of the longitudinal rib 1217. An outer edge 1223 of the longitudinal rib 1217 may be recessed at a first location 1219 where the longitudinal rib 1217 crosses the flow path 1210 compared to a second location 1221 between successive turns of the flow path 1210. In other configurations, an outer edge 1223 of the longitudinal rib 1217 may be generally straight or smooth along a majority of or entire length of the longitudinal rib 1217, without any recesses along the outer edge 1223 of the longitudinal rib 1217.

Each longitudinal rib 1217 may begin at or be spaced apart from the first end 1204 of the elongate body 1202. The longitudinal ribs 1217 may converge toward each other at or near the first end 1204 of the tampon 1200. For example, the longitudinal ribs 1217 may follow a tapered or conical portion 1207 of the first end 1204. The longitudinal ribs 1217 may be bunched or compressed together at the first end 1204 to form the insertion tip of the tampon 1200. The bunched insertion tip may include a reduced diameter compared to the remainder of the tampon. For example, the bunched insertion tip may extend from the tapered portion 1207 of the tampon 1200. The convergence of the longitudinal ribs 1217 at the insertion tip guides fluid toward the core and traps fluid in the absorbent internal fibers within the elongate body 1202. This prevents fluid from running down the outer surface 1225 of the tampon 1200.

Each longitudinal rib 1217 may terminate at or be spaced apart from the second end 1206 of the elongate body 1202. Each longitudinal rib 1217 may terminate at or near a transition 1205 between a lateral surface and a bottom surface of the tampon 1200. For example, the longitudinal 1217 may extend around the transition 1205 and across at least a portion of the bottom surface of the tampon 1200. Each rib 1217 may extend at least a majority or substantially the entire length of the elongate body 1202.

A thickness of each longitudinal rib 1217, measured in a radial direction, may be general constant along a length of the longitudinal rib 1210. In other configurations, the thickness of the longitudinal rib 1217 may vary along the length of the longitudinal rib 1217. For example, the thickness of the longitudinal rib 1217 may be thicker at the first location 1219 where the longitudinal rib 1217 crosses the flow path 1210 compared to the second location 1221 between successive turns of the flow path 1210.

Upon expansion and/or saturation of the tampon, the elongate body 1202 may expand toward the outer edge 1223 of the longitudinal ribs 1217, thereby reducing a thickness of the longitudinal ribs 1217 projecting from the outer surface 1225. In some configurations, the outer surface 1225 of the elongate body 1202 may become flush with the plurality of longitudinal ribs 1217. For example, the tampon 1200 may include a first expanded state in which the longitudinal ribs 1217 have a first thickness, and a second expanded state in which the longitudinal ribs 1217 have a second thickness, different from the first thickness. In some configurations, the second thickness may be negligible or zero with the longitudinal ribs 1217 no longer being visible or present. In some configurations, the elongate body 1202 may not expand beyond the outer edges 1223 of the longitudinal ribs 1217. The longitudinal ribs 1217 may preserve a maximum circumference of the tampon 1200, while making portions of the tampon 1200 between the longitudinal ribs 1217 smaller for insertion. As the tampon 1200 expands, the flow path 1210 may become more prominent, for example, a width of the flow path 1210 may increase.

The longitudinal ribs 1217 may be circumferentially spaced apart by at least about 2 mm, at least about 4 mm, or at least about 6 mm (e.g. 2-4 mm, 3-5 mm, 4-6 mm and numerical values within those ranges). The space between successive longitudinal ribs 1217 may be constant or varied around the circumference of the elongate body 1202. Each rib 1217 may extend from the outer surface 1225 of the elongate body 1202 by no more than about 5 mm or no more than about 3 mm (e.g., 2-4 mm, 2.5-4.5 mm, 3-5 mm and numerical values within those ranges). The width of each rib 1217, measured in a circumferential direction, may be less than or equal to about 5 mm (e.g., 3-5 mm, 1-3 mm, 0.5-2 mm and numerical values within those ranges) or less than or equal to about 2 mm. The width of each rib 1217 may be substantially constant along a length of the elongate body 1202. The ribs 1217 may extend continuously from the first end 1204 to the second end 1206. Each rib 1217 may begin within 10 mm (or within 5 mm, or within 2 mm) from the first end 1204 and terminate within 10 mm (or within 5 mm or within 2 mm) from the second end 1206.

The elongate body 1202 may include a core surrounded by an outer layer. The outer layer may conform to the shape of the core. The outer layer extends along at least a partial length or an entire length of the core. The outer layer may cover one or both ends of the core and form an outer surface of the insertion end and/or removal end. In other configurations, one or both ends of the core may be exposed from the outer layer. In tampons 1200 with the outer layer, the longitudinal ribs 1217 may be formed substantially entirely or entirely of the outer layer, although in some configurations, fibers of the internal core may form an internal portion of the longitudinal ribs 1217.

The core can be a fibrous structure made with different quantities of fibers depending on absorbency. The core may have between about 1.0 g and about 2.5 g of fibers. For example, the core may be 1.5 g for a regular absorbency tampon or 2 g for a super absorbency tampon. The fibrous construction of the core may be generally constant or vary along a length of the core. For example, the core may include fewer fibers or less density at the insertion end compared to the removal end.

The outer layer may include a nonwoven material, which may be the same or different from the core material of the elongate body 1202. The nonwoven material may be natural or synthetic, including but not limited to materials such as polyestradiol phosphate, cotton, organic cotton and other organic materials, unbleached cotton and other materials, polypropylene or polyethylene based nonwoven materials, filament nonwoven fabric, synthetic fibers, rayon, viscose, lyocell, bamboo, foam, modified cross-section fiber, trilobal cellulosic fiber, or a blend including any of the aforementioned materials. The outer layer may be sealed at the first end 1204 such that the core is completely covered at the first end 1204. The overwrap layer may be applied to the fibrous core using different methods, for example by embossing a calendared sliver of the fibrous absorbent material with the outer layer, inserting the core into a tube-shaped outer layer, or applying the outer layer as a flat sheet to the core.

Referring back to FIG. 8, in one example, the outer or cover layer may be provided over the elongate body in step 462. The rectangle of the outer layer material may be formed into a tube by sealing two edges of the rectangle together. One end of the tube may be sealed, for example with a heat seal. The elongate body may be inserted into the outer layer tube.

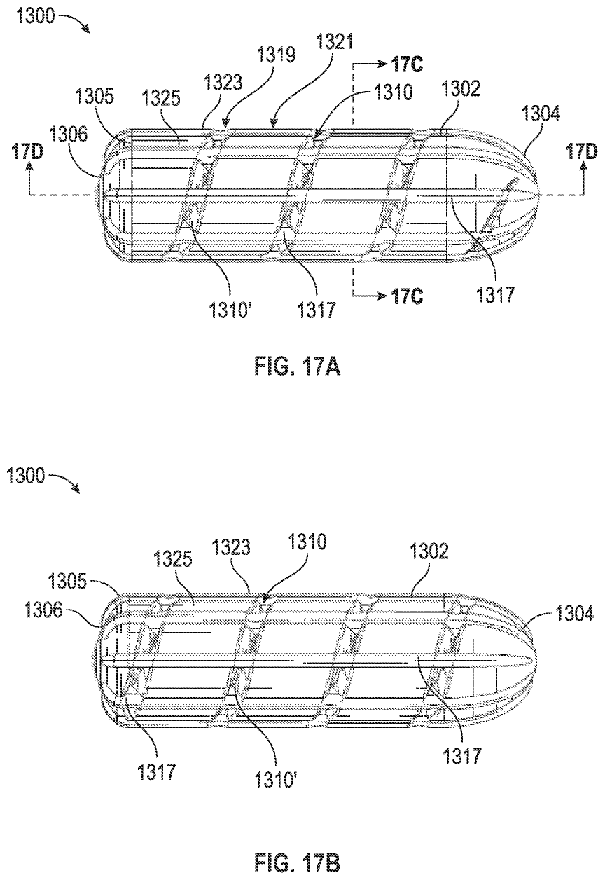

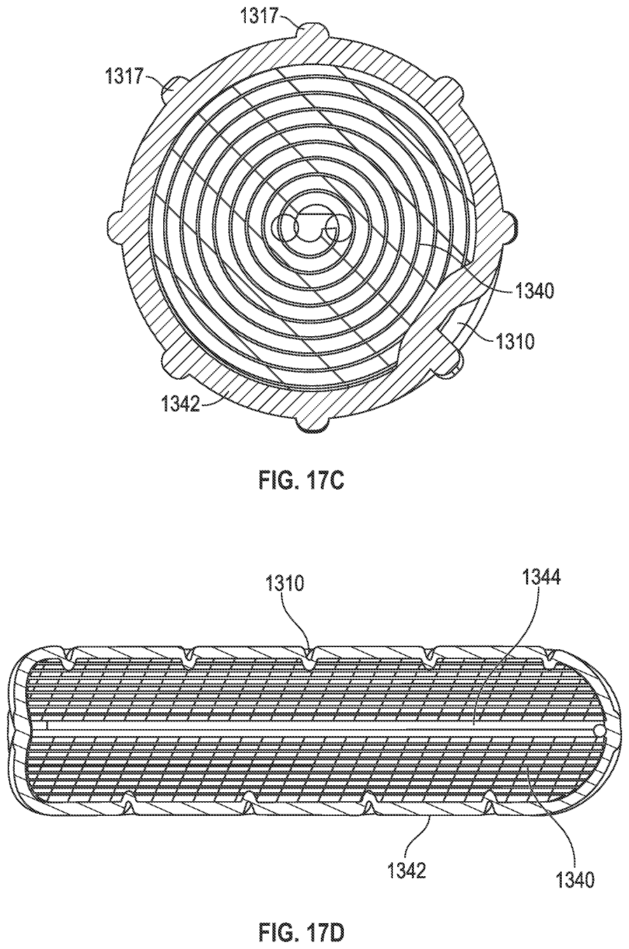

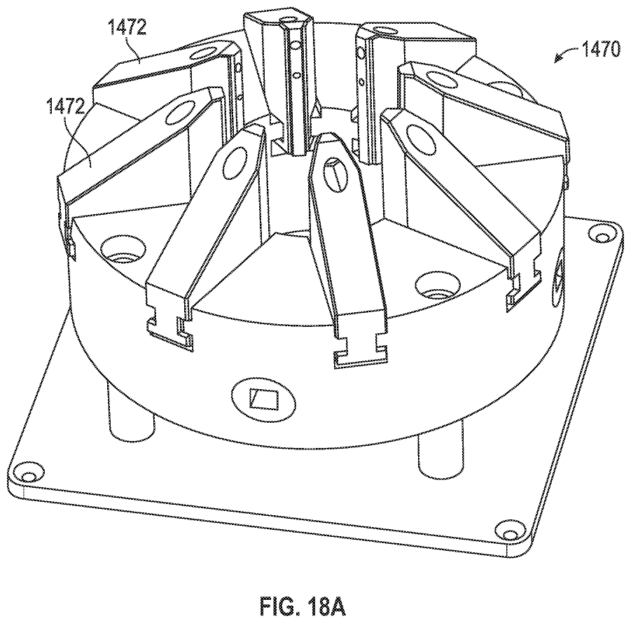

FIGS. 17A and 17B show another tampon 1300 that is similar to the tampon 1200 except as described below. As mentioned above, the longitudinal ribs may converge toward each other at or near the first end of the tampon. In FIGS. 16A and 16B, the longitudinal ribs 1217 converge at the first end 1204 to form the insertion tip of the tampon 1200. Alternatively, the ends of the longitudinal ribs 1317 may be spaced apart from the insertion tip of the tampon 1300 as shown in FIGS. 17A and 17B. The first end 1304 of the tampon 1300 may include a rounded surface to facilitate insertion. The first end 1304 may be covered by the outer layer.

After forming the elongate body using any of the methods described above, the flow path may be molded into the outer surface of the elongate body, for example using a radial compression mold. The localized compression along the flow path extends the length of the flow path and adds more surface area. Because there is a longer path and more contact with the fibers, the rate of fluid flow is slowed in the longitudinal direction. The slower fluid flow provides more time for the elongate body to absorb fluid, promotes full saturation of the tampon, and delays possible leakage. The fibrous construction of the longitudinal ribs may be less dense than the compressed fibers along the flow path.

FIG. 17C illustrates a transverse cross-section of the tampon 1300. As illustrated, the core 1340 is rolled up, for example in a swiss roll design, to form the elongate body 1302. The longitudinal ribs 1317 are formed substantially from the outer layer 1342. As shown in FIG. 17D, the withdrawal string 1344 extends through substantially the entire elongate body 1302.

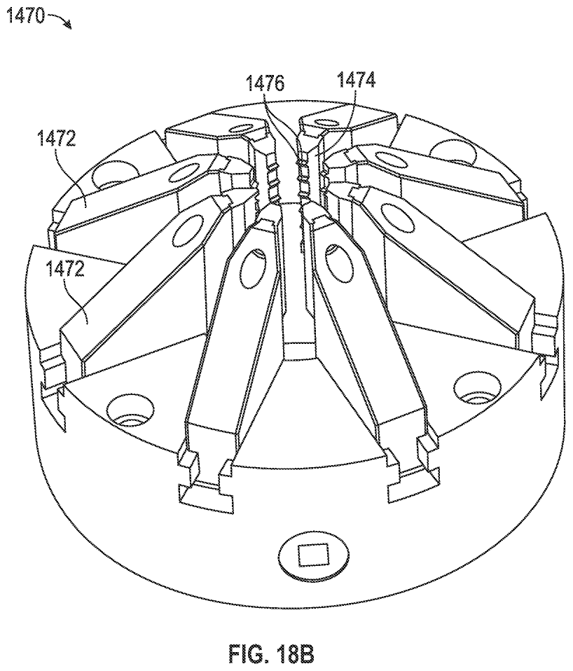

FIGS. 18A and 18B provide an example of a compression mold 1470 to create the tampon 1200, although a similar mold could be used to create the tampon 1300. The compression mold 1470 includes one or more mold segments 1472 disposed circumferentially around the mold 1470. Each mold segment 1472 may be tapered toward a center of the mold 1470. Each segment 1472 may span between 30 degrees and 180 degrees of the elongate body, for example between 45 degrees and 90 degrees of the elongate body. In use, the formed tampon may be disposed at the center of the one or more mold segments 1472. As the one or more mold segments 1472 move radially inward toward the elongate body, e.g., from FIG. 18A to FIG. 18B, the flow path is formed in the outer surface of the elongate body. This compression process may be manual or automated.

As illustrated in FIG. 18B, each mold segment 1472 may include a body portion 1474 and one or more projections 1476 projecting from the body portion 1374. The body portion 1474 may be constructed of aluminum. Each projection 1476 may form a negative of a flow segment of the flow path to be created on the elongate body. Together, the projections form the generally spiral flow path. When the mold segments 1472 are compressed inward, space remains between adjacent mold segments 1472. The longitudinal ribs are extruded through the space between adjacent mold segments 1472. The upper ends of the body portions 1474 converge toward each other to cinch the insertion end of the tampon.

Although a particular manufacturing method is described with respect to FIGS. 18A and 18B, the tampons described herein may be manufactured using other methods. For example, after the elongate body is formed, the elongate body may be twisted to form the flow path prior to forming the longitudinal ribs.

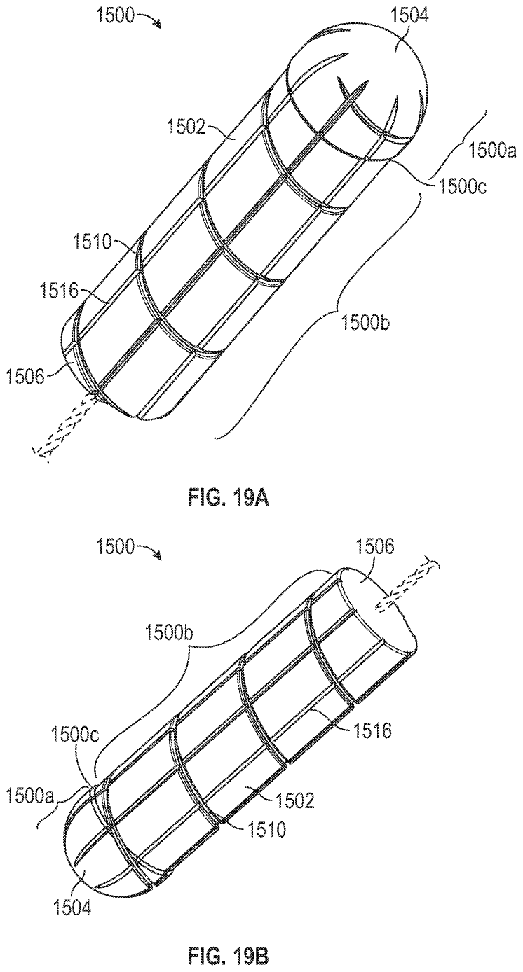

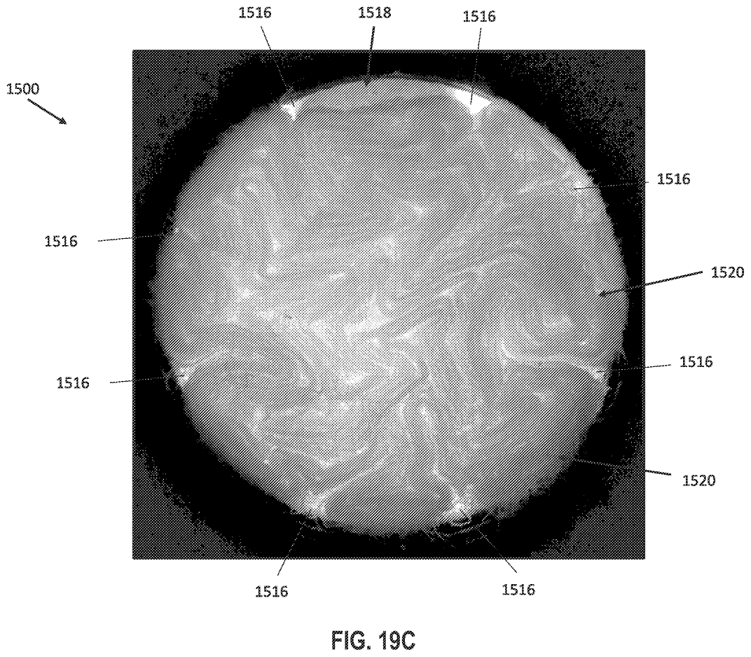

With reference to FIGS. 19A-19B, another tampon 1500 is shown. The tampon 1500 can include any of the features described above with respect to tampon 300.

As illustrated, the tampon 1500 can include an elongate body 1502 having an insertion or first end 1504 and a removal or second end 1506. The elongate body 1502 can include a fibrous core having a plurality of internal folds as shown in FIG. 19C. The elongate body 1502 can include a first region 1500a at the first end 1504, a second region 1500b at the second end 1506, and a transition region 1500c therebetween. There may be a step-like profile or undercut at the transition region 1500c.

As illustrated, the first region 1500a can be generally dome-shaped, while the second region 1500b can be generally cylindrical. A total length of the tampon 1500 can be at least about 40 mm and/or less than or equal to about 50 mm, for example between 35 mm and 45 mm or between 40 mm and 50 mm, such as between 40 mm and 45 mm. A length of the first region 1500 a can be less than a length of the second region 1500 b, for example less than or equal to about 30% (or less than or equal to about 25%, less than or equal to about 20%, or less than or equal to about 15%) of a length of the second region 1500 b.

A diameter of the first region 1500a and/or the second region 1500b can be at least about 5 mm and/or less than or equal to 20 mm, for example between 5 mm to 10 mm, between 10 mm to 15 mm, or between 15 mm to 20 mm. The first region 1500 a can have a first diameter, and the second region 1500 b can have a second diameter. The first diameter can be different compared to the second diameter. The first diameter can be at least about 5% or at least about 10% greater than the second diameter. The difference in diameter may be no greater than 2 mm or no greater than 1 mm. In other configurations, the first region 1500a may be generally cone-shaped or include a diameter that is no greater than a diameter of the second region 1500b.

A weight of the tampon 1500a, including any removal string, can be less than or equal to 5 g, less than or equal to 4 g, less than or equal to 3 g, or less than or equal to 2 g. The tampon 1500 has a volume to absorb a liquid quantity of at least 5 g and/or less than or equal to 10 g, for example between 6 g and 9 g.

The tampon 1500 can have a spiral flow path 1510 including any of the features of the above-described spiral flow paths. As illustrated, the spiral flow path 1500 is a spiral recess but could be a spiral projection. The spiral flow path 1510 can extend at least one complete turn (or at least two, or at least three, or at least four turns) around a longitudinal axis of the elongate body 1502. The spiral flow path 1510 can be a right-handed spiral flow path or a left-handed spiral flow path. The spiral flow path 1510 can extend from the first region 1500a to the second region 1500b. A first end of the spiral flow path 1510 may be at the first end 1504 or spaced apart from the first end 1504 but within the first region 1500a. A first turn of the spiral flow path 1510, beginning at the first end of the spiral flow path, may traverse the transition region 1500c. A pitch of the spiral flow path 1500 may be less than or equal to about 2.0 cm or less than or equal to about 1.5 cm or less than or equal to about 1.0 cm. A length of the first region 1500a may be less than or equal to a pitch of the spiral flow path 1510. In some embodiments, a second end of the spiral flow path 1510 extends to the second end 1506, but in other embodiments, may be spaced apart from the second end 1506.

The spiral flow path 1510 can be a generally continuous flow path or at least appear to be a generally continuous flow path. For example, as explained in further detail below, the spiral flow path 1510 can be formed by a plurality of projections on each of a plurality of mold segments. Each of the plurality of projections forms an indentation (also referred to herein as a well) on the elongate body 1502. Depending on the arrangement of the mold segments, the indentations may be sufficiently aligned to form a generally continuous flow path. The generally continuous flow path may be formed by a plurality of discrete indentations in a spiral pattern. The plurality of discrete indentations may be sufficiently close with the longitudinal grooves 1516, described further below, to allow fluid to flow from one indentation to a circumferentially adjacent indentation or between the longitudinal grooves 1516 and the indentations. The plurality of discrete indentations may be sufficiently close together to allow a continuous flow around the spiral flow path. The generally continuous flow path may have a substantially uniform depth along a length of the spiral flow path, for example any portion of the spiral flow path can have a depth within 10% or within 5% of a mean depth of the spiral flow path. In some arrangements, the depth of the spiral flow path 1510 may vary along a length of the spiral flow path. For example, regions of the spiral flow path 1510 formed between different mold segments may be shallower compared to regions of the spiral flow path formed by the projections on the mold segments 1510.

The tampon 1500 can optionally have one or more longitudinal flow paths. As illustrated, the longitudinal flow paths are longitudinal recesses or grooves 1516 extending in a direction of an axis traversing the first end 1504 and the second end 1506, but as described earlier could be longitudinal ribs. Each longitudinal groove 1516 may extend from the first region 1500a to the second region 1500b. Each longitudinal groove 1516 may begin at or near the first end 1504 of the elongate body 1502 and terminate at or near the second end 1506 of the elongate body 1502. The longitudinal grooves 1516 may be spaced apart from each other around a circumference of the elongate body 1502. Each longitudinal groove 1516 may cross successive turns of the spiral flow path 1510. The intersections between the longitudinal grooves 1516 and the spiral flow path 1510 can slow the flow of fluid from the first end 1504 toward the second end 1506. Thus, a high number of intersections may be desirable. For example, the tampon can include at least 24 intersections, at least 28 intersections, at least 32 intersections, or at least 36 intersections. The combination of longitudinal grooves 1516 and the spiral flow path 1510 can create recesses along at least 15% (or at least 20%, or at least 25%, or at least 30%) of a surface area of the elongate body 1502. The overall length of the flow paths can slow the travel of fluid. The spiral flow path 1510 can intersect the longitudinal groove 1516 which can enable damming of the fluid and can give more opportunities for the fluid to be absorbed.

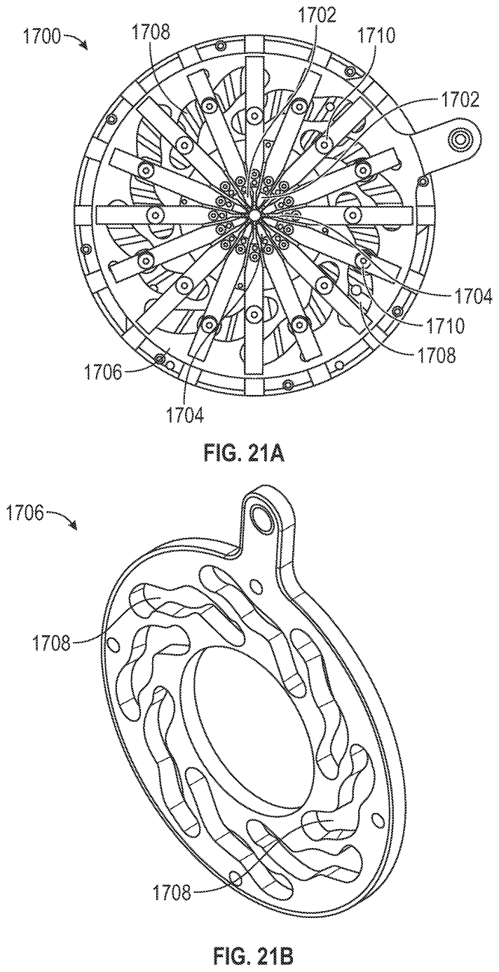

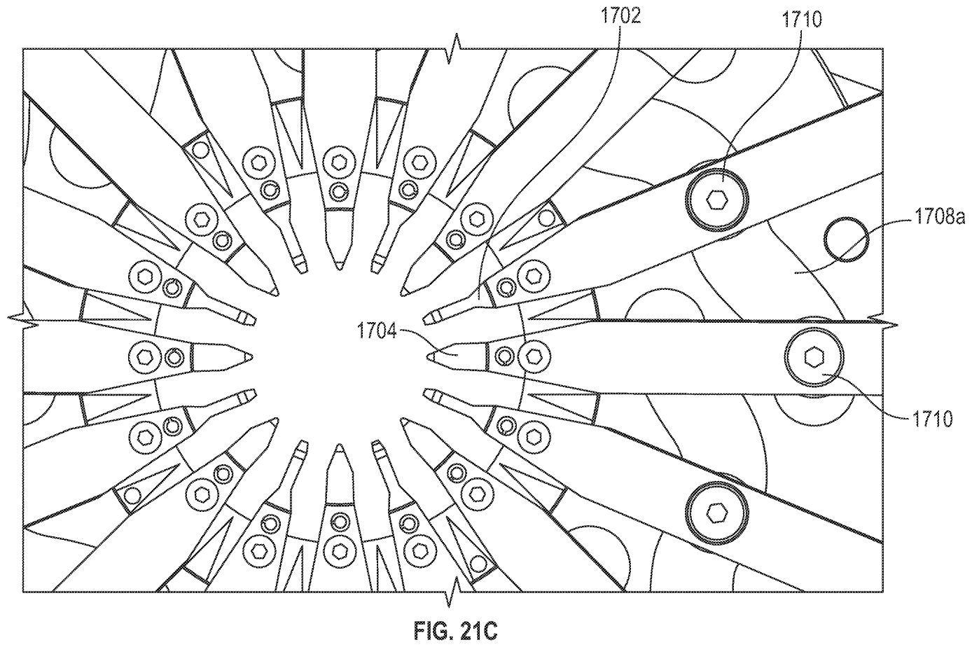

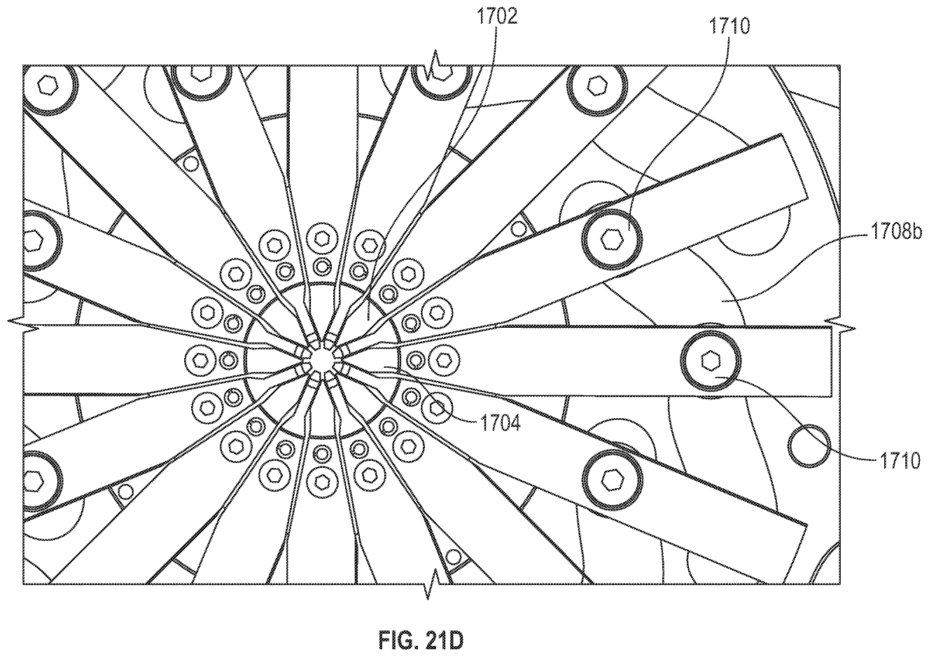

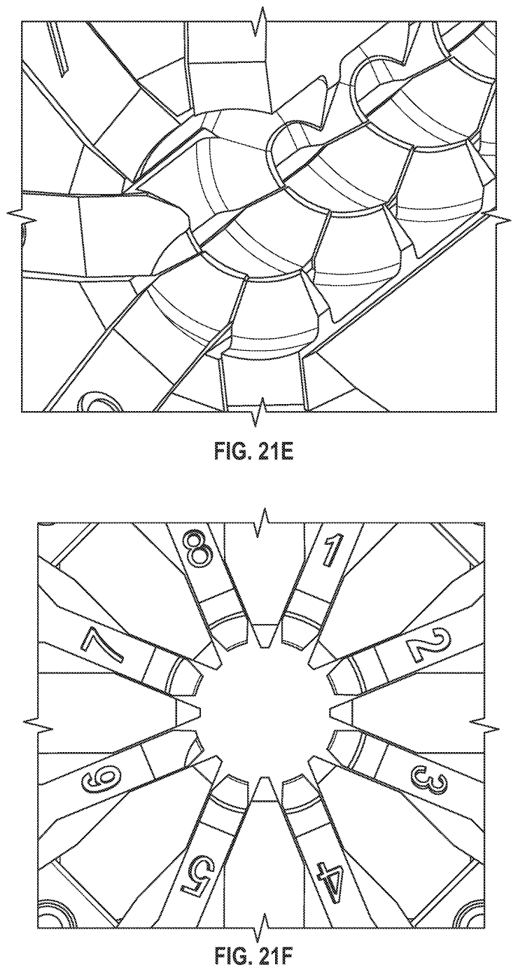

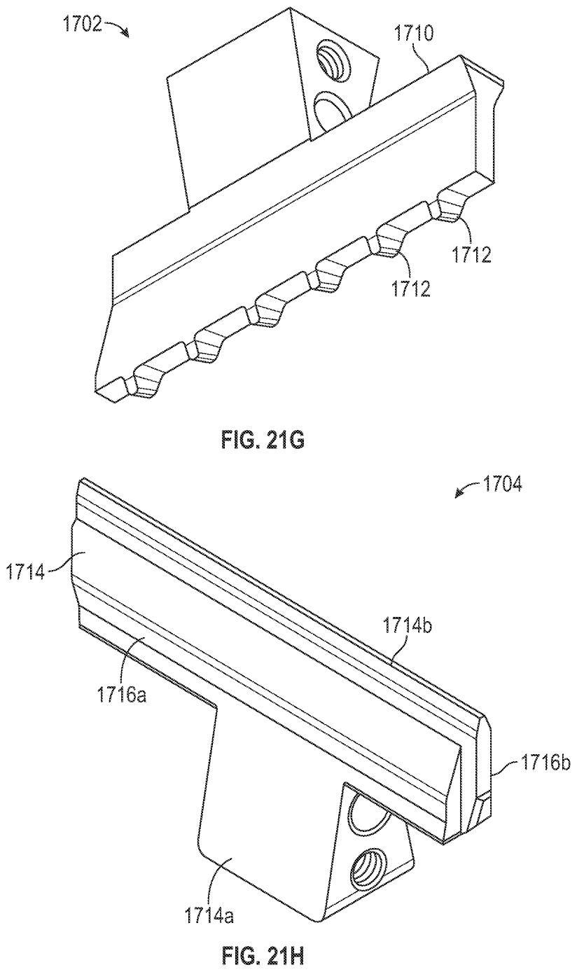

Each longitudinal groove 1516 may be defined by a first wall extending in a radial direction and a second wall extending in a radial direction. In some arrangements, the first wall and the second wall are angled toward each other from an outer surface of the tampon toward an apex of the respective longitudinal groove 1516. The first wall and the second wall generally extend along a radius of the tampon 1500. A length of the first wall, measured from the outer surface of the tampon toward the apex, is substantially the same as a length of the second wall, measured from the outer surface of the tampon toward the apex. When viewed in cross-section, the longitudinal groove 1516 forms a generally triangular-shaped cutout in an outer surface of the tampon 1500a. The first wall and the second wall can be generally straight or planar walls formed, for example, by the first surface 1616a and the second surface 1616b of jaw 1604 (see FIG. 201). In other words, the first wall and the second wall are non-arcuate walls.

Each longitudinal groove 1516 may be circumferentially spaced apart by at least about 2 mm, at least about 4 mm, or at least about 6 mm (e.g. 2-4 mm, 3-5 mm, 4-6 mm and numerical values within those ranges). The space between successive longitudinal groove 1516 may be constant or varied around the circumference of the elongate body 302. A depth of the longitudinal grooves 1516 can be less than or equal to about 2 mm, less than or equal to about 1.5 mm, or less than or equal to about 1 mm. The depth of the longitudinal grooves 1516 may be less than a depth of the spiral flow path 1510. In other words, the spiral path 1510 extends deeper into the elongate body 1502 than the longitudinal grooves 1516. The width at an opening of the longitudinal grooves 1516 may be less than a width at the opening of the spiral flow path 1510.

As shown in FIG. 19C, the flow paths in the elongate body 1502 can form a plurality of wells 1518. Each well 1518 can be formed by a segment of the spiral flow path 1510 and extend from one of the longitudinal grooves 1516 to an adjacent longitudinal groove 1516. The wells 1518 can be in direct contact with two adjacent longitudinal grooves 1516. Circumferentially adjacent wells 1518 can be longitudinal spaced apart from each other. Viewed another way, the longitudinal grooves 1518 can form a plurality of lobes 1520 between adjacent longitudinal grooves 1518. Each lobe 1520 can include a plurality of wells 1518 longitudinal spaced apart from each other along a length of the lobe 1520. All of the plurality of wells can be spaced apart from the insertion end of the tampon. For example, in some configurations, all of the plurality of wells can be located in the second region 1500b such that no wells are located in the first region 1500a. In other configurations, there may be at least one well in the first region 1500a. At any given position along a length of the tampon, there may be only one well 1518 around the circumference of the tampon. For example, when viewed in cross-section through one of the wells 1518, there is only one well 1518 (see, e.g., top of FIG. 19C). Each well 1518 can have a depth of at least 5% (or at least 10% or at least 15%) and/or less than or equal to 20% of a diameter of the elongate body, where depth is measured from an outer surface towards a core of the elongate body. For example, each well 1518 can have a depth of at least about 1.0 mm and/or less than or equal to about 3.0 mm, for example at least 1.5 mm, or at least 2.0 mm. The opening of the well (at the outer surface) can have a height in a longitudinal direction of at least about 1.0 mm and/or less than or equal to about 3.0 mm, for example at least about 1.5 mm, or at least 2.0 mm. The height of the opening of the well can be equal to the depth of the well, but in other configurations the two measurements can be different. The height of the well can taper from the outer surface towards the core.

The wells 1518 allow fluid to flow toward the core of the elongate body 1502. As fluid flows down the longitudinal grooves 1516, the fluid will pool in the wells 1518. This will slow or stop further flow of fluid along the longitudinal grooves 1516. The fluid will encounter a plurality of wells 1518 along the length of any one of the longitudinal grooves 1516. The plurality of wells 1518 give the core more time to absorb the fluid and reduce the likelihood of possible leakage before full saturation. In use, the wells 1518 provide a gap between the vaginal wall and the absorbent material of the tampon to provide a space for fluid to dwell and provide more time for absorption into the tampon. As blood pools in any given well 1518, blood may start to coagulate within the wells 1518, which can also slow the travel of fluid along the longitudinal grooves 1516.

The high number of wells 1518 interrupts the overall flow of fluid from the insertion end to the removal end and gives the tampon more time to absorb the fluid. For example, the tampon 1500 can include at least 24 wells, at least 28 wells, or at least 32 wells.

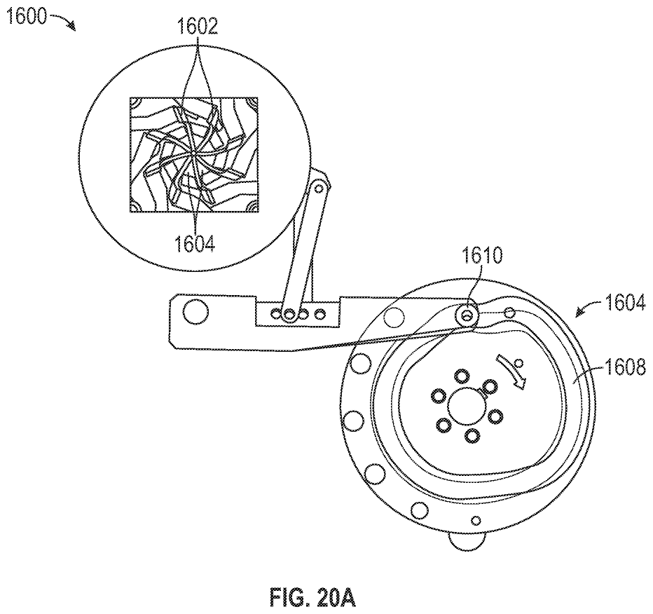

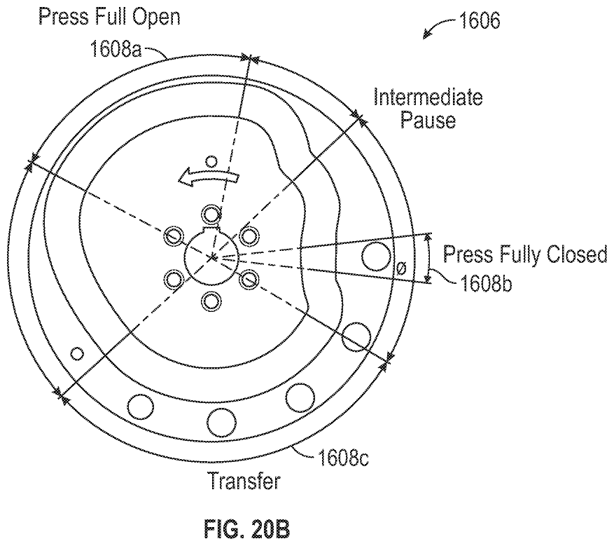

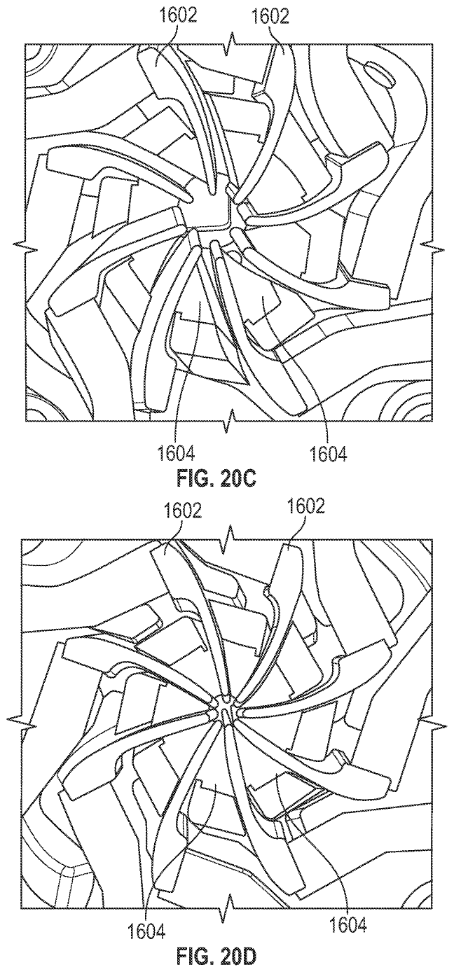

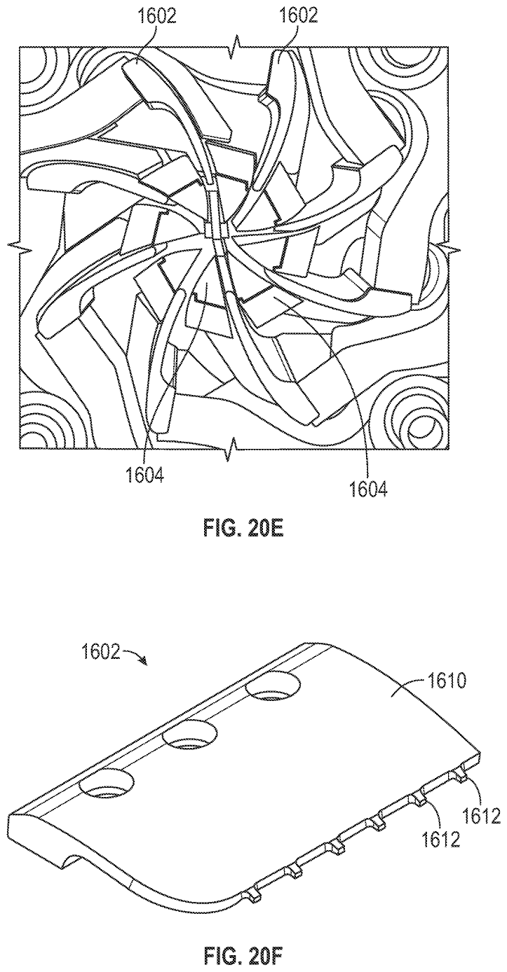

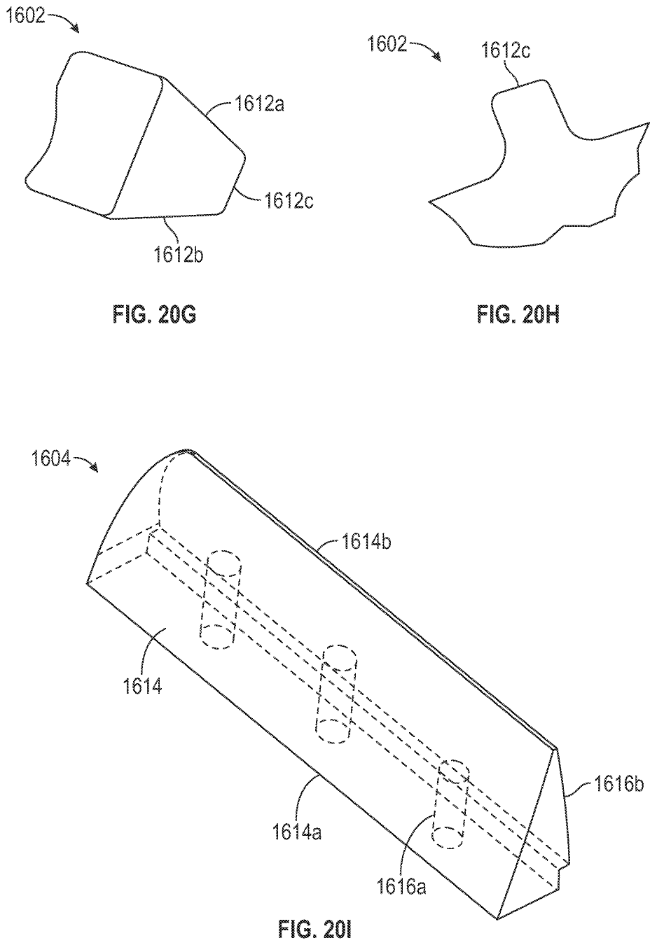

FIG. 20A illustrates a tampon press 1600 that can be used to form the tampon 1500. As illustrated, the tampon press 1600 includes a first set of shaping jaws 1602 and a second set of transfer jaws 1604. The first set of shaping jaws 1602 can be used to form a first flow path. The first flow path may be formed by a plurality of wells, for example wells 1518 described above. The second set of transfer jaws 1604 can be used to form a second flow path. The first flow path can be different from the second flow path. For example, the first flow path may be equivalent to the spiral flow path 1510 made up of the plurality of wells 1518, and the second flow path may be equivalent to the longitudinal grooves 1516. Each of the jaws 1602, 1604 can be mounted to a radial arm. The path of the first set of shaping jaws 1602 and the second set of transfer jaws 1604 can be driven by the external cam 1606. The external cam 1606 can be displaced from the jaws 1602, 1604 by one or more lever arms. One of the lever arms can include a cam follower 1610 that follows the cam path 1608 in the external cam 1606.

FIG. 20B illustrates an enlarged view of the external cam 1606. The cam path 1608 can form a general ring-shape such that the external cam 1606 only needs to move in one direction (e.g., clockwise or counterclockwise) to drive the jaws 1602, 1604 around a full cycle. The radius of curvature of cam path 1608 varies with different stages of press operation. A first section 1608a of the cam path 1608 can represent a stage where the first set of jaws 1602 and the second set of jaws 1604 are fully open (see FIG. 20C). In the fully open position, the first set of jaws 1602 and the second set of jaws 1604 move radially outward to allow a blank to be inserted into the press. The first section 1608 a can extend at least 15% and/or less than or equal to about 30% of a length of a cam path 1608, for example at least 20% or less than or equal to about 25% of a length of the cam path 1608. An intermediate pause phase can occur as the first set of jaws 1602 and the second set of jaws 1604 move radially inward. The intermediate pause can allow for the first set of jaws 1602 to form parallel adjacent walls around the first set of jaws 1604 before the first set of jaws 1602 and the second set of jaws 1604 contact the blank. The first set of jaws 1602 forming parallel adjacent walls around the first set of jaws 1604 can avoid the jaws from pinching the fibers of the blank when contacting the blank. Pinching of the fibers can cause external ribs to be formed which can be avoided with the intermediate pause.

A second section 1608b of the cam path 1608 can represent a stage where the first set of jaws 1602 and the second set of jaws 1604 are fully closed (see FIG. 20D). In the fully closed position, the first set of jaws 1602 and the second set of jaws 1604 move radially inward to form flow path(s) in the blank. In this position, the first set of jaws 1602 may extend further radially inward relative to the second set of jaws 1604. In other arrangements, the innermost edges of the first set of jaws 1602 and the second set of jaws 1604 may be circumferentially aligned. The second section 1608a can extend less than or equal to about 10% or less than or equal to about 5% of a length of the cam path 1608.

A third section 1608c of the cam path 1608 can represent a stage where the first set of jaws 1602 move radially outward relative to the second set of jaws 1604 (see FIG. 20E) to enable transfer of the tampon out of the tampon press. The third section 1608c can extend at least about 20% and/or less than or equal to about 40% of a length of the cam path 1608, for example at least 25% and less than or equal to about 35% of a length of the cam path 1608. In some cases, a second intermediate pause can occur as the first set of jaws 1602 move radially outward relative to the second set of jaws. The second intermediate pause can pause the first set of jaws 1602 to keep first set of jaws 1602 and the second set of jaws 1604 close together.

In some arrangements, both the spiral flow path 1510 and the longitudinal grooves 1516 are formed in the fully closed stage. In other arrangements, the spiral flow path 1510 is formed in the fully closed stage, and the longitudinal grooves are formed in the transfer stage, an intermediate stage between the fully open stage and the fully closed stage or in an intermediate stage between the fully closed stage and the transfer stage.

The radius of curvature in the first section 1608a can be the greatest radius of curvature in the cam path 1608. The radius of curvature of the second section 1608b can be the smallest radius of curvature in the cam path 1608. The radius of curvature of the third section 1608c can be between the radius of curvature of the first section 1608a and the second section 1608b.

An arc length of the third section 1608c can be greater than an arc length of the first section 1608a and the second section 1608b. An arc length of the first section 1608a can be greater than an arc length of the second section 1608b.

As illustrated in FIG. 20A, the first set of shaping jaws 1602 and the second set of transfer jaws 1604 can be disposed circumferentially around the press. The first set of shaping jaws 1602 and the second set of transfer jaws 1604 can be disposed in alternating order. In other words, each shaping jaws 1602 is positioned between two transfer jaws 1604, and each transfer jaw 1604 is disposed between two shaping jaws 1602. The first set of shaping jaws 1602 may include a set of eight shaping jaws 1602, but the number of shaping jaws 1602 can vary depending on the dimensions of the shaping jaws 1602 and/or tampon. The number of shaping jaws 1602 may be the same or different than the number of transfer jaws 1604.

FIG. 20F shows an example shaping jaw 1602. The shaping jaw 1602 can include a body portion 1610. The body portion 1610 can include a first side configured to be mounted to a radial arm (see FIG. 20A) and a second side having a plurality of projections 1612 for shaping the tampon blank. Each of the plurality of projections 1612 can form one of the wells 1518. The body portion 1610 can have a generally curved body shape in a direction from the radial arm towards the projections 1612. A width of the body portion 1610, measured from the first side to the second side, can be greater than a depth of the body portion 1610, measured in a circumferential direction, in use. The width of the body portion 1610 can be less than a length of the body portion 1610, measured in a direction extending through the plurality of projections 1612.

Each projection 1612 can form a negative of a segment of a flow path to be created in the tampon. The plurality of projections 1612 can be spaced apart along a length of the body portion 1610. Since each projection 1612 forms a portion of a spiral flow path, the projection 1612 may be generally angled when viewing the projection from an innermost surface of the projection 1512.

FIG. 20G illustrates a bottom view of one of the projections 1612. As shown, each projection 1612 can have a first side 1612a and a second side 1612b tapered toward each other at an angle between 30 degrees and 60 degrees, for example about 45 degrees. The end surface 1612c of the projection can form a blunt or flat edge. In other arrangements, the end surface 1612 has a concave profile. Corners formed between the end surface 1612c and the first side 1612a or second side 1612b can be rounded, chamfered, rounded, or sharp. A depth of the projection, measured in the direction of the first side 1612a or second side 1612b, can be less than or equal to 5 mm, less than or equal to 4 mm, less than or equal to 3 mm, or less than or equal to 2 mm.

FIG. 20H illustrates a side view of one of the projections 1612, perpendicular to the view shown in FIG. 20G. Corners formed between the projection 1612 and the body portion 1610 can be rounded. A width of the end surface 1612c of the projection 1612 can be less than or equal to 5 mm, less than or equal to 4 mm, less than or equal to 3 mm, or less than or equal to 2 mm.

When the shaping jaws 1602 are used to form the spiral flow path 1510, each of the shaping jaws 1602 can be different to form different sections of the spiral flow path 1510. For example, the position of the plurality of projections 1612 on each of the shaping jaws 1602 may differ.

FIG. 201 shows an example of a transfer jaw 1604. All the transfer jaws can be identical. The transfer jaw 1604 includes a body portion 1614. The body portion 1614 can include a first side 1614a configured to be mounted to a radial arm (see FIG. 20A) and a second side 1614 b facing the tampon, in use. A depth of the jaw 1604, measured from the first side 1614a to the second side 1614b, can be less than or equal to 5 mm, less than or equal to 4 mm, less than or equal to 3 mm, or less than or equal to 2 mm. In some embodiments, the second side 1614b can be shaped to form a second type of flow path, for example the longitudinal grooves 1516. In other embodiments, the second side 1614 does not form any flow paths, but merely holds the tampon in place during the transfer process. During the press operation described above, the transfer jaw may be pressed into the elongate body at a depth of at least 1.0 mm, at least 1.5 mm, at least 2.0 mm, or at least 2.5 mm. The resulting longitudinal groove may have a depth that is less than the depth at which the transfer jaw is pressed into the elongate body.

As shown, the second side 1614b forms a straight edge. The body portion 1614a can include a first surface 1616a and a second surface 1616b angled toward each other to form the straight edge. In this manner, the profile of the jaw 1604 is generally triangular. The first surface 1616a and the second surface 1616b may be angled toward each other at an angle of less than or equal to about 60 degrees, or less than or equal to about 50 degrees, or less than or equal to about 45 degrees.

FIG. 21A illustrates another tampon press 1700 that can be used to form the tampon 1500. Tampon press 1700 can include a first set of shaping jaws 1702 and a second set of transfer jaws 1704. Each jaw 1702, 1704 can be mounted to a radial arm. The first set of shaping jaws 1702 can be used to form a first flow path. The second set of transfer jaws 1704 can be used to form a second flow path. The first flow path can be different from the second flow path. For example, the first flow path may be equivalent to the spiral flow path 1510, and the second flow path may be equivalent to the longitudinal grooves 1516.

The path of the first set of shaping jaws 1702 and the second set of transfer jaws 1704 can be driven by the internal cam 1706. The internal cam 1706 can be in-line with the jaws 1702, 1704. Each radial arm can include a cam follower 1610 that follows a cam path 1708 in the internal cam 1606. As shown in FIG. 21A, each cam path 1708 corresponds to one of the shaping jaws 1702 and one of the transfer jaws 1704. There can be an equal number of shaping jaws 1702, transfer jaws 1704, and cam paths 1708.

FIG. 21B illustrates an enlarged view of the internal cam 1706. The radius of curvature of cam path 1708 varies with different stages of press operation. Unlike the cam path 1608 illustrated above in FIG. 20B, none of the cam paths 1708 form a ring. Instead, the internal cam 1706 moves in both the clockwise and counterclockwise direction relative to the jaws 1702, 1704 to drive the jaws 1702, 1704 along a full cycle.

As shown in FIG. 21C, when the jaws 1702, 1704 are in a first section 1708a of the cam path 1708, the first set of jaws 1702 and the second set of jaws 1704 are fully open. The transfer jaw 1704 is at or near an end of the cam path 1708 and the shaping jaw 1702 is in a mid-section of the cam path 1708. In the fully open position, the first set of jaws 1702 and the second set of jaws 16704 move radially outward to allow a blank to be inserted into the press.

As shown in FIGS. 21D and 21E, when the jaws 1702, 1704 are in a second section 1708b of the cam path 1708, the first set of jaws 1702 and the second set of jaws 1704 are fully closed. The shaping jaw 1702 is at or near the opposite end of the cam path 1708 and the transfer jaw is in a mid-section of the cam path 1708. In the fully closed position, the first set of jaws 1702 and the second set of jaws 1704 move radially inward to form flow path(s) in the blank. As shown in FIGS. 21D and 21E, innermost surfaces of the first set of jaws 1702 project further inward than innermost surfaces of the second set of jaws 1704. In this position, the first set of jaws 1702 is able to form the generally continuous spiral flow path 1510 in the tampon. In other arrangements, the innermost surface of the first set of jaws 1702 and the innermost surface of the second set of jaws 1704 may be circumferentially aligned in the fully closed position.

Rotating the internal cam 1706 in the opposite direction causes the jaws 1702, 1704 to transition from the fully closed position back to the fully open position. During this transition, the first set of jaws 1702 move radially outward relative to the second set of jaws 1704 (see FIG. 21F). In this transfer position, the tampon can be transferred out of the press.

In some arrangements, both the spiral flow path 1510 and the longitudinal grooves 1516 are formed in the fully closed stage. In other arrangements, the spiral flow path 1510 is formed in the fully closed stage, and the longitudinal grooves are formed in the transfer stage, an intermediate stage between the fully open stage and the fully closed stage, or in an intermediate stage between the fully closed stage and the transfer stage.

As illustrated in FIG. 21A, the first set of shaping jaws 1702 and the second set of transfer jaws 1704 can be disposed circumferentially around the press. The first set of shaping jaws 1702 and the second set of transfer jaws 1704 can be disposed in alternating order. In other words, each shaping jaws 1702 is positioned between two transfer jaws 1704, and each transfer jaw 1704 is disposed between two shaping jaws 1602. The first set of shaping jaws 1702 may include a set of eight shaping jaws 1602, but the number of shaping jaws 1702 could be varied depending on the dimensions of the shaping jaws 1702 and/or tampon. The number of shaping jaws 1702 may be the same or different than the number of transfer jaws 1704.

FIG. 21G shows an example shaping jaw 1702. The shaping jaw includes a body portion 1710. The body portion 1610 can include a first side configured to be mounted to a radial arm (see FIG. 21A) and a second side having a plurality of projections 1712 for shaping the tampon blank. The body portion 1710 can have a generally tapered shape in a direction from the radial arm towards the projections 1712. A width of the body portion 1710, measured from the first side to the second side, can be greater than a depth of the body portion 1710, measured in a circumferential direction, in use. The width of the body portion 1710 can be less than a length of the body portion 1710, measured in a direction extending through the plurality of projections 1712.

Each projection 1712 can form a negative of a segment of a flow path to be created in the tampon. The plurality of projections 1712 can be spaced apart along a length of the body portion 1710. Since each projection 1712 forms a portion of a spiral flow path, the projection 1712 may be generally angled when viewing the projection from an innermost surface of the projection 1712.

When the shaping jaws 1702 are used to form the spiral flow path 1510, each of the shaping jaws 1702 can be different to form different sections of the spiral flow path 1510. For example, the position of the plurality of projections 1712 on each of the shaping jaws 1702 may differ. The projections 1712 can include any of the features of the projections 1612.

FIG. 21H shows an example of a transfer jaw 1704. All the transfer jaws can be identical. The transfer jaw includes a body portion 1714. The body portion can include a first side 1714a configured to be mounted to a radial arm (see FIG. 21A) and a second side 1714b facing the tampon, in use. In some embodiments, the second side 1714b can be shaped to form a second type of flow path, for example the longitudinal grooves 1516. In other embodiments, the second side 1714 does not form any flow paths, but merely holds the tampon in place during the transfer process.

As shown, the second side 1714b forms a straight edge. The body portion 1714a can include a first surface 1716a and a second surface 1716b angled toward each other to form the straight edge. In this manner, the profile of the jaw 1704 is generally triangular. The first surface 1716a and the second surface 1716b may be angled toward each other at an angle of less than or equal to about 60 degrees, or less than or equal to about 50 degrees, or less than or equal to about 45 degrees.

In several embodiments, a single tampon may include a combination of any of the flow paths and ribs described above. In any of the above-described tampons, the tampon may have the described flow path or ribs to the exclusion of any other flow paths, particularly without any longitudinal grooving, substantially longitudinal grooving, or grooving that makes less than a full turn around a circumference of the elongate body. For example, the tampon may have a single, continuous flow path to the exclusion of any other flow paths. The single, continuous flow path may have a single starting point at or near the insertion end of the elongate body and a single termination point at or near the removal end of the elongate body. In any of the above-mentioned external flow paths, the flow path may terminate at the removal end of the elongate body without extending across the end face of the removal end (e.g., the flattened base). As another example, the tampon may include the longitudinal ribs without the spiral flow path or any other flow paths.

In several embodiments, the above-described tampons improve the efficiency of absorbency compared to similar sized tampons on the market. As a result, the total time that elapses prior to leakage is longer compared to similar sized tampons on the market. For women with a heavier menstrual flow, the tampon designs described herein may allow a user to use a smaller sized tampon or tampon having a lower absorbency rating because the tampon is more likely to reach full saturation before leaking. The smaller sized tampon may be less noticeable to the user and reduce discomfort during removal.

As described above, the annular or spiral flow paths described herein lengthen the total flow path for menstrual material. For women with more viscous menses or blood clots, the flow path provides greater travel for the viscous menstrual material and gives the tampon additional time to absorb the menstrual material. A total length of the flow path can be at least about 100 mm, for example between about 150 mm and about 250 mm or between about 175 mm and about 225 mm. The total length of the flow path can be at least two or three times greater than a length of the elongate body, for example between about three and about five. This reduces the likelihood of possible leakage before full saturation of tampon, so women do not have to continually worry about checking their tampons.

Since the tampons described herein are capable of absorbing a greater amount of fluid before leaking, less frequent replacement of tampons is needed. This lowers the total number of products needed during a menstrual cycle. In several embodiments, the tampons include external flow paths that conform to the rugae along the vaginal walls to prevent displacement of the tampon during active movement. Any of the above-mentioned tampons may also include a sustainable material, such as bamboo that provides higher absorbency and antibacterial properties.

Any of the tampons described herein can be inserted using an applicator or digitally inserted. As the tampon is inserted, the tampon may generally expand in an axial and/or radial direction. For tampons inserted using the applicator, the applicator may be made of a plastic or cardboard material. The applicator may include an outer tubular body and an inner plunger. The outer tubular body may have a smooth surface and/or a rounded end to facilitate insertion. An insertion end of the outer tubular body has an opening for deployment of the tampon. The inner plunger may be slidably disposed within the outer tubular body to facilitate ejection of the tampon. The inner plunger may take the form of an inner tubular body.

Any of the above-described tampons may be provided in a kit. For example, the kit may include a plurality of any of the above-mentioned tampons or a combination of any of the above-mentioned tampons. For any given type of tampon within the kit, there may be tampons having a different absorbency rating. For example, the kit may include one or more tampons of the same type (e.g., absorbent material and/or flow path design) having a light absorbency, regular absorbency, super absorbency, super plus absorbency, and/or ultra-absorbency rating to allow the user to accommodate different levels of flow during a menstrual cycle. Within any given type of tampon, the absorbency may be adjusted based on a mass of the tampon and/or the dimensions of the flow path. The change in absorbency only impacts the weight, circumference, and/or size of the tampon, not the structure or proportions of the design. The plurality of tampons may be sufficient to last a user for one menstrual cycle or multiple menstrual cycles, for example three menstrual cycles. The kit may include at least two to eight tampons per day of the menstrual cycle. The kit may additionally include one or more liners, pads, and/or period underwear.

The tampons described herein may have one or more regions of fluid impervious or fluid wicking regions. In some embodiments, the tampon comprises at least one region that is less or more absorbent than other regions. In some embodiments, the core of the tampon is comprised of filler material or other material that is different (in material and/or absorbency) than the rest of the tampon. Although certain tampons have been described herein in connection with menstrual cycle, the tampons described herein can be used for wound healing, nose bleeds, surgical sites, fecal incontinence, or absorption of any other bodily fluids. In some embodiments, the tampon can be a plug or other body for absorbing bodily fluid. The tampon may take on a shape other than the elongate shape illustrated herein. For example, the tampon may have a conical, bulbous, flattened, or other shaped body. The tampon may be used to deliver medications to the body. The medicated portion may be a separate portion on and/or in the tampon, or the tampon may be coated or embedded with the medication. The medication may be helpful for analgesia, dysmenorrhea, blood flow and clotting, antimicrobial activity, etc. In some embodiments, the tampon is designed to expand (e.g., self-expand, expand upon release of constraint, expand upon exposure to body temperature, expand upon fluid exposure, etc.) The ranges disclosed herein also encompass any and all overlap, sub-ranges, and combinations thereof. Language such as “up to,” “at least,” “greater than,” “less than,” “between,” and the like includes the number recited. Numbers preceded by a term such as “about,” “approximately,” or “near” include the recited numbers and should be interpreted based on the circumstances (e.g., as accurate as reasonably possible under the circumstances, for example ±1%, ±5%, ±10%, ±15%, etc.). For example, “about 2 mm” includes “2 mm.” Phrases preceded by a term such as “substantially” or “generally” include the recited phrase and should be interpreted based on the circumstances (e.g., as much as reasonably possible under the circumstances). For example, “generally longitudinal” includes “longitudinal.” Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Although certain embodiments and examples have been described herein, it will be understood by those skilled in the art that many aspects of the tampons shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

1. A method of manufacturing a tampon, the method comprising:
inserting a tampon blank into a tampon press in an open configuration, the tampon press comprising a first set of jaws and a second set of jaws different from the first set of jaws;
moving the first set of jaws and the second set of jaws radially inward toward a closed configuration to shape the tampon blank,
forming a plurality of wells in the tampon blank using the first set of jaws;
forming a plurality of longitudinal recesses in the tampon blank using the second set of jaws; and
moving the first set of jaws radially outward relative to the second set of jaws toward a transfer configuration to eject the shaped tampon blank from the press.

2. The method of claim 1, further comprising forming a spiral recess in the tampon blank using the first set of jaws.

3. The method of claim 1, further comprising driving the first set of jaws and the second set of jaws along a single cam path to move between the open configuration, the closed configuration, and the transfer configuration.

4. The method of claim 1, further comprising driving the first set of jaws and the second set of jaws along a plurality of cam paths to move between the open configuration, the closed configuration, and the transfer configuration, each one of the plurality of cam paths associated with one of the first set of jaws and one of the second set of jaws.

5. The method of claim 1, wherein the first set of jaws and the second set of jaws are moved toward the closed configuration from the open configuration.

6. The method of claim 1, wherein the first set of jaws is moved toward the transfer configuration from the closed configuration.

7. The method of claim 1, wherein moving the first set of jaws and the second set of jaws radially inward toward the closed configuration comprises simultaneously moving the first set of jaws and the second set of jaws.

8. A method of manufacturing a tampon, the method comprising:
inserting a tampon blank into a tampon press in an open configuration, the tampon press comprising a first set of jaws and a second set of jaws different from the first set of jaws;
moving the first set of jaws and the second set of jaws radially inward toward a closed configuration to shape the tampon blank into a shaped tampon, and
moving the first set of jaws radially outward relative to the second set of jaws toward a transfer configuration to eject the shaped tampon blank from the tampon press.

9. The method of claim 8, further comprising forming a spiral recess in the tampon blank using the first set of jaws.

10. The method of claim 8, further comprising driving the first set of jaws and the second set of jaws along a single cam path to move between the open configuration, the closed configuration, and the transfer configuration.

11. The method of claim 8, further comprising driving the first set of jaws and the second set of jaws along a plurality of cam paths to move between the open configuration, the closed configuration, and the transfer configuration, each one of the plurality of cam paths associated with one of the first set of jaws and one of the second set of jaws.

12. The method of claim 8, wherein the first set of jaws and the second set of jaws are moved toward the closed configuration from the open configuration.

13. The method of claim 8, wherein the first set of jaws is moved toward the transfer configuration from the closed configuration.

14. The method of claim 10, wherein moving the first set of jaws and the second set of jaws radially inward toward the closed configuration comprises simultaneously moving the first set of jaws and the second set of jaws.

15. A method of manufacturing a tampon, the method comprising:
inserting a tampon blank into a tampon press in an open configuration, the tampon press comprising a first set of jaws and a second set of jaws different from the first set of jaws;
moving the first set of jaws and the second set of jaws radially inward toward a closed configuration to shape the tampon blank,
forming a plurality of wells in the tampon blank using the first set of jaws; and
forming a plurality of longitudinal recesses in the tampon blank using the second set of jaws.

16. The method of claim 15, further comprising forming a spiral recess in the tampon blank using the first set of jaws.

17. The method of claim 15, further comprising driving the first set of jaws and the second set of jaws along a single cam path to move between the open configuration, the closed configuration, and a transfer configuration.

18. The method of claim 15, further comprising driving the first set of jaws and the second set of jaws along a plurality of cam paths to move between the open configuration, the closed configuration, and a transfer configuration, each one of the plurality of cam paths associated with one of the first set of jaws and one of the second set of jaws.

19. The method of claim 15, wherein the first set of jaws and the second set of jaws are moved toward the closed configuration from the open configuration.

20. The method of claim 15, wherein the first set of jaws is moved toward a transfer configuration from the closed configuration.

21. The method of claim 15, wherein moving the first set of jaws and the second set of jaws radially inward toward the closed configuration comprises simultaneously moving the first set of jaws and the second set of jaws.

22.-86. (canceled)
